SEQUENCE LISTING (38 sequences)

SEQ ID NO: 1 - DNA, 306 bp, Artificial Sequence
  Description of Artificial Sequence Synthetic polynucleotide
ctgactcagccgtcctcggtgtcagcaaacccaggaggaaccgtcgagatcacctgctccgggagtagtggcagctatggctggtatcagcagaagtcacctggcagtgcccctgtcactgtgatctattacaacgacaagagaccctcggacatcccttcacgattctccggttccaaatccggctccacagccacattaaccatcactggggtccaagccgaggacgaggctgtctatttctgtgggagtgaagacagcagctatgttggtgtatttggggccgggacaaccctgaccgtccta

SEQ ID NO: 2 - protein, 102 aa, Artificial Sequence
  Description of Artificial Sequence Synthetic polypeptide
LTQPSSVSANPGGTVEITCSGSSGSYGWYQQKSPGSAPVTVIYYNDKRPSDIPSRFSGSKSGSTATLTITGVQAEDEAVYFCGSEDSSYVGVFGAGTTLTVL

SEQ ID NO: 3 - DNA, 372 bp, Artificial Sequence
  Description of Artificial Sequence Synthetic polynucleotide
gccgtgacgttggacgagtccgggggcggcctccagacgcccggaggaacgctcagcctcgtctgcaagggctccgggttcaccttcaggagttatgccctggagtgggtgcgccaggcacccggcaaggggctggaatacgtcgcgggtattagcagcagtggcagtggcacaaactacgggtcggcggtgaagggccgtgccaccatctcgagggacaacgggcagagcacagtgaggctgcagctgaacaacctcagggctgaggacaccggcacctactactgcgccaaaagtgcttacggttatgttgatgcttacggcatcgacgcatggggccacgggaccgaagtcatcgtctcctccactagt

SEQ ID NO: 4 - protein, 124 aa, Artificial Sequence
  Description of Artificial Sequence Synthetic polypeptide
AVTLDESGGGLQTPGGTLSLVCKGSGFTFRSYALEWVRQAPGKGLEYVAGISSSGSGTNYGSAVKGRATISRDNGQSTVRLQLNNLRAEDTGTYYCAKSAYGYVDAYGIDAWGHGTEVIVSSTS

SEQ ID NO: 5 - DNA, 309 bp, Artificial Sequence
  Description of Artificial Sequence Synthetic polynucleotide
ctgactcagccgtcctcggtgtcagcaaacccgggagaaaccgtcaagatcacctgctccgggggtggtaggtggtatggctggtaccagcagaagtctcctggcagtgcccctgtcactctgatccatgctaataccaaaagaccctcgaacatcccttcacgattctccggttccctatccggctccacaagcacattaaccatctctggggtccaagccgaggacgaggctgtctatttctgtgggagtggagacagcagcactgatagtggtatatttggggccgggacaaccctgaccgtccta

SEQ ID NO: 6 - protein, 103 aa, Artificial Sequence
  Description of Artificial Sequence Synthetic polypeptide
LTQPSSVSANPGETVKITCSGGGRWYGWYQQKSPGSAPVTLIHANTKRPSNIPSRFSGSLSGSTSTLTISGVQAEDEAVYFCGSGDSSTDSGIFGAGTTLTVL

SEQ ID NO: 7 - DNA, 381 bp, Artificial Sequence
  Description of Artificial Sequence Synthetic polynucleotide
gccgtgacgttggacgagtccgggggcggcctccagacgcccggaggagcgctcagcctcgtctgcaaggcctccgggttctccttcagtgaccgtgcaatgcactgggtgcgacaggcacccggcaaggggctggagtgggtcgcgggtatttacagcagtggtagatacacaggctacgggtcggcggtgaagggccgtgccaccatctcgagggacaacgggcagagcacagtgaggctgcagctgaacaacctcagggctgaggacaccggcacctactactgcgccaaagctggtagtatttactgtgggtatgctgatgttgcttgcatcgacgcgtggggccacgggaccgaagtcatcgtctcctccactagt

SEQ ID NO: 8 - protein, 127 aa, Artificial Sequence
  Description of Artificial Sequence Synthetic polypeptide
AVTLDESGGGLQTPGGALSLVCKASGFSFSDRAMHWVRQAPGKGLEWVAGIYSSGRYTGYGSAVKGRATISRDNGQSTVRLQLNNLRAEDTGTYYCAKAGSIYCGYADVACIDAWGHGTEVIVSSTS

SEQ ID NO: 9 - DNA, 189 bp, Homo sapiens
tcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcatgccggccagcggcggggggcgcagtgcacacgagggggctggat

SEQ ID NO: 10 - protein, 63 aa, Homo sapiens
SALSNSIMYFSHFVPVFLPAKPTTTPAPRPPTPAPTIASQPLSLRPEACRPAAGGAVHTRGLD

SEQ ID NO: 11 - DNA, 213 bp, Mus sp.
cctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgcccc

SEQ ID NO: 12 - protein, 71 aa, Mus sp.
PKLFWALVVVAGVLFCYGLLVTVALCVIWTNSRRNRLLQSDYMNMTPRRPGLTRKPYQPYAPARDFAAYRP

SEQ ID NO: 13 - DNA, 339 bp, Mus sp.
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 112 aa, Mus sp.
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1533 bp, Artificial Sequence
  Description of Artificial Sequence Synthetic polynucleotide
ctgactcagccgtcctcggtgtcagcaaacccaggaggaaccgtcgagatcacctgctccgggagtagtggcagctatggctggtatcagcagaagtcacctggcagtgcccctgtcactgtgatctattacaacgacaagagaccctcggacatcccttcacgattctccggttccaaatccggctccacagccacattaaccatcactggggtccaagccgaggacgaggctgtctatttctgtgggagtgaagacagcagctatgttggtgtatttggggccgggacaaccctgaccgtcctaggtcagtcctctagatcttccggcggtggtggcagctccggtggtggcggttccgccgtgacgttggacgagtccgggggcggcctccagacgcccggaggaacgctcagcctcgtctgcaagggctccgggttcaccttcaggagttatgccctggagtgggtgcgccaggcacccggcaaggggctggaatacgtcgcgggtattagcagcagtggcagtggcacaaactacgggtcggcggtgaagggccgtgccaccatctcgagggacaacgggcagagcacagtgaggctgcagctgaacaacctcagggctgaggacaccggcacctactactgcgccaaaagtgcttacggttatgttgatgcttacggcatcgacgcatggggccacgggaccgaagtcatcgtctcctccactagtgcggccgcagaacaaaaactcatctcagaagaggatctgaatggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcatgccggccagcggcggggggcgcagtgcacacgagggggctggatcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccctcgagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 511 aa, Artificial Sequence
  Description of Artificial Sequence Synthetic polypeptide
LTQPSSVSANPGGTVEITCSGSSGSYGWYQQKSPGSAPVTVIYYNDKRPSDIPSRFSGSKSGSTATLTITGVQAEDEAVYFCGSEDSSYVGVFGAGTTLTVLGQSSRSSGGGGSSGGGGSAVTLDESGGGLQTPGGTLSLVCKGSGFTFRSYALEWVRQAPGKGLEYVAGISSSGSGTNYGSAVKGRATISRDNGQSTVRLQLNNLRAEDTGTYYCAKSAYGYVDAYGIDAWGHGTEVIVSSTSAAAEQKLISEEDLNGVTVSSALSNSIMYFSHFVPVFLPAKPTTTPAPRPPTPAPTIASQPLSLRPEACRPAAGGAVHTRGLDPKLFWALVVVAGVLFCYGLLVTVALCVIWTNSRRNRLLQSDYMNMTPRRPGLTRKPYQPYAPARDFAAYRPLERVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1545 bp, Artificial Sequence
  Description of Artificial Sequence Synthetic polynucleotide
ctgactcagccgtcctcggtgtcagcaaacccgggagaaaccgtcaagatcacctgctccgggggtggtaggtggtatggctggtaccagcagaagtctcctggcagtgcccctgtcactctgatccatgctaataccaaaagaccctcgaacatcccttcacgattctccggttccctatccggctccacaagcacattaaccatctctggggtccaagccgaggacgaggctgtctatttctgtgggagtggagacagcagcactgatagtggtatatttggggccgggacaaccctgaccgtcctaggtcagtcctctagatcttccggcggtggtggcagctccggtggtggcggttccgccgtgacgttggacgagtccgggggcggcctccagacgcccggaggagcgctcagcctcgtctgcaaggcctccgggttctccttcagtgaccgtgcaatgcactgggtgcgacaggcacccggcaaggggctggagtgggtcgcgggtatttacagcagtggtagatacacaggctacgggtcggcggtgaagggccgtgccaccatctcgagggacaacgggcagagcacagtgaggctgcagctgaacaacctcagggctgaggacaccggcacctactactgcgccaaagctggtagtatttactgtgggtatgctgatgttgcttgcatcgacgcgtggggccacgggaccgaagtcatcgtctcctccactagtgcggccgcagaacaaaaactcatctcagaagaggatctgaatggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcatgccggccagcggcggggggcgcagtgcacacgagggggctggatcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccctcgagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 515 aa, Artificial Sequence
  Description of Artificial Sequence Synthetic polypeptide
LTQPSSVSANPGETVKITCSGGGRWYGWYQQKSPGSAPVTLIHANTKRPSNIPSRFSGSLSGSTSTLTISGVQAEDEAVYFCGSGDSSTDSGIFGAGTTLTVLGQSSRSSGGGGSSGGGGSAVTLDESGGGLQTPGGALSLVCKASGFSFSDRAMHWVRQAPGKGLEWVAGIYSSGRYTGYGSAVKGRATISRDNGQSTVRLQLNNLRAEDTGTYYCAKAGSIYCGYADVACIDAWGHGTEVIVSSTSAAAEQKLISEEDLNGVTVSSALSNSIMYFSHFVPVFLPAKPTTTPAPRPPTPAPTIASQPLSLRPEACRPAAGGAVHTRGLDPKLFWALVVVAGVLFCYGLLVTVALCVIWTNSRRNRLLQSDYMNMTPRRPGLTRKPYQPYAPARDFAAYRPLERVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 312 bp, Mus sp.
atcgagttcatgtacccaccaccctaccttgacaatgaacggagtaacggaactatcattcacatcaaggaaaagcatctttgccacacacaatctagtccaaaactcttttgggcattggtcgttgtggctggggtgcttttctgttatggacttcttgtgaccgtagcactctgtgtcatctggacaaatagccgccgaaaccgcggcgggcagtccgactacatgaacatgactcctagacgccctggactgactcgcaaaccctatcaaccttatgctcccgctagggacttcgcagcctataggccc

SEQ ID NO: 20 - protein, 104 aa, Mus sp.
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRP

SEQ ID NO: 21 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 112 aa, Homo sapiens; identical to SEQ ID NO: 14

SEQ ID NO: 23 - DNA, 651 bp, Artificial Sequence
  Description of Artificial Sequence Synthetic polynucleotide
atcgagttcatgtacccaccaccctaccttgacaatgaacggagtaacggaactatcattcacatcaaggaaaagcatctttgccacacacaatctagtccaaaactcttttgggcattggtcgttgtggctggggtgcttttctgttatggacttcttgtgaccgtagcactctgtgtcatctggacaaatagccgccgaaaccgcggcgggcagtccgactacatgaacatgactcctagacgccctggactgactcgcaaaccctatcaaccttatgctcccgctagggacttcgcagcctataggcccagagccaaattttcccgatctgctgagactgccgccaatctccaggacccaaatcaattgtttaacgaactgaaccttggacggcgggaagagtttgatgttttggagaagaagcgcgcacgcgaccctgagatgggtggcaagcagcaacgccgacgaaaccctcaagagggtgtttacaatgccctgcaaaaggacaagatggcagaggcttatagcgaaataggaacaaagggggaacggagacgaggaaagggacatgatggactttttcaggggctctccacagccacaaaggatacattcgacgccttgcacatgcaaacccttgctcctaga

SEQ ID NO: 24 - protein, 217 aa, Artificial Sequence
  Description of Artificial Sequence Synthetic polypeptide
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 25 - DNA, 30 bp, Artificial Sequence
  Description of Artificial Sequence Synthetic oligonucleotide
gaacaaaaactcatctcagaagaggatctg

SEQ ID NO: 26 - protein, 10 aa, Artificial Sequence
  Description of Artificial Sequence Synthetic peptide
EQKLISEEDL

SEQ ID NO: 27 - DNA, 20 bp, Artificial Sequence
  Description of Artificial Sequence Synthetic primer
actcattgtggctgtggaga

SEQ ID NO: 28 - DNA, 20 bp, Artificial Sequence
  Description of Artificial Sequence Synthetic primer
ttgttcatctcggagcctgt

SEQ ID NO: 29 - DNA, 20 bp, Artificial Sequence
  Description of Artificial Sequence Synthetic primer
ctggggatgtctgtagctca

SEQ ID NO: 30 - DNA, 20 bp, Artificial Sequence
  Description of Artificial Sequence Synthetic primer
ctgtgaagtctcctctccgg

SEQ ID NO: 31 - DNA, 20 bp, Artificial Sequence
  Description of Artificial Sequence Synthetic primer
gattgcggggttgtatctgg

SEQ ID NO: 32 - DNA, 20 bp, Artificial Sequence
  Description of Artificial Sequence Synthetic primer
gctttctttcagggacagcc

SEQ ID NO: 33 - DNA, 20 bp, Artificial Sequence
  Description of Artificial Sequence Synthetic primer
caactcccactcttccacct

SEQ ID NO: 34 - DNA, 20 bp, Artificial Sequence
  Description of Artificial Sequence Synthetic primer
gagttgggatagggcctctc

SEQ ID NO: 35 - DNA, 720 bp, Artificial Sequence
  Description of Artificial Sequence Synthetic polynucleotide
ctgactcagccgtcctcggtgtcagcgaacccgggagaaaccgtcgagatcacctgctccgggggtggcagctactatggctggtaccagcagaagtctcctggcagtgcccctgtcactgtgatctatagcaacaacaagagaccctcggacatcccttcacgattctccggttccaaatccggctccacaagcacattaaccatcactggggtccaagccgacgacgaggctgtctattactgtgggagctacgacagcaatgctggtatatttggggccgggacaaccctgaccgtcctaggtcagtcctctagatcttccggcggtggtggcagctccggtggtggcggttccgccgtgacgttggacgagtccgggggcggcctccagacgcccggaggagcactcagcctcgtctgcaaggcctccgggttcaccttcagcagttatgccatgggttggatgcgccaggcacccggcaaggggctggacttcgtcgctgaaattagcggcagtggcactagcacatactacgggccggcggtgaagggccgtgccaccatctcgagggacaacgggcggagcacagtgaggctgcagctgaacaacctcagggctgaggacaccggcacctacttctgcacgagaggtggtggtgctggtagttacatcgacgcatggggccacgggaccgaagtcatcgtctcctccactagt

SEQ ID NO: 36 - protein, 240 aa, Artificial Sequence
  Description of Artificial Sequence Synthetic polypeptide
LTQPSSVSANPGETVEITCSGGGSYYGWYQQKSPGSAPVTVIYSNNKRPSDIPSRFSGSKSGSTSTLTITGVQADDEAVYYCGSYDSNAGIFGAGTTLTVLGQSSRSSGGGGSSGGGGSAVTLDESGGGLQTPGGALSLVCKASGFTFSSYAMGWMRQAPGKGLDFVAEISGSGTSTYYGPAVKGRATISRDNGRSTVRLQLNNLRAEDTGTYFCTRGGGAGSYIDAWGHGTEVIVSSTS

SEQ ID NO: 37 - DNA, 753 bp, Artificial Sequence
  Description of Artificial Sequence Synthetic polynucleotide
atgcttctcctggtgacaagccttctgctctgtgagttaccacacccagcattcctcctgatcccagagcagaagctgatcagcgaggaggacctgattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 38 - protein, 251 aa, Artificial Sequence
  Description of Artificial Sequence Synthetic polypeptide
MLLLVTSLLLCELPHPAFLLIPEQKLISEEDLIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0082305 A1
(43) Pub. Date: Mar. 14, 2024

(54) ANTI-C4D CHIMERIC ANTIGEN RECEPTOR REGULATORY T CELLS AND USES THEREOF

(71) Applicants: Seoul National University R&DB Foundation, Seoul (KR); Seoul National University Hospital, Seoul (KR); The Asan Foundation, Seoul (KR)

(72) Inventors: Jaeseok YANG, Seoul (KR); Sun-Kyung LEE, Seoul (KR); Joon Young JANG, Seoul (KR); Junho CHUNG, Seoul (KR); Jerome HAN, Seoul (KR); Nara SHIN, Seoul (KR); Hyori KIM, Seoul (KR)

(21) Appl. No.: 18/262,303

(22) PCT Filed: Jan. 20, 2022
(86) PCT No.: PCT/IB2022/050495

§ 371 Date: Jul. 20, 2023

Related U.S. Application Data
(60) Provisional application No. 63/139,617, filed on Jan. 20, 2021.

(51) Int. Cl. A61K 35/17; A61K 39/00; A61P 37/06; C07K 16/28; C12N 5/0783

(52) U.S. Cl. CPC A61K 35/17; A61K 39/4611; A61K 39/4631; A61K 39/46433; A61P 37/06; C07K 16/2833; C12N 5/0637; A61K 2239/13; A61K 2239/17; A61K 2239/21

(57) ABSTRACT

Antibody-mediated rejection (ABMR) is one of the main obstacles to successful transplantation, including ABO blood group-incompatible (ABOi) transplantation. C4d deposition is a marker of ABMR and is also found in most ABOi allograft tissues. Described herein are anti-C4d CAR Tregs that suppress ABMR in ABOi allografts. Anti-C4d CAR Tregs prepared by retroviral transduction of CAR into CD62L +CD4 +CD25 +Tregs, expressed Foxp3, CD25, CTLA-4, LAP, and GITR to similar extents as non-transduced Tregs. Anti-C4d CAR Tregs were activated by specific binding to C4d and suppressed in vitro T cell proliferation as well as non-transduced Tregs. Furthermore, adoptive transfer of anti-C4d CAR Tregs significantly prolonged mouse ABOi heart allograft survival (P<0.05).

### CROSS REFERENCE TO RELATED APPLICATIONS

This application claims priority to U.S. Provisional Application No. 63/139,617, filed Jan. 20, 2021, which is hereby incorporated by reference in its entirety for all purposes.

### REFERENCE TO A “SEQUENCE LISTING” SUBMITTED AS ASCII TEXT FILE

The instant application contains a Sequence Listing which has been submitted electronically in ASCII format and is hereby incorporated by reference in its entirety. Said ASCII copy, created on Jan. 19, 2022, is named 107568-1292165_SL.txt and is 39,541 bytes in size.

### BACKGROUND

ABO blood group-incompatible (ABOi) transplantation has been developed to overcome the serious problem of donor organ shortage in transplantation.^{1,2} However, antibody-mediated rejection (ABMR) remains as the main limitation to successful ABOi transplantation. Introduction of desensitization treatment consisting of plasmapheresis or immunoadsorption with rituximab and strong maintenance immunosuppressants such as tacrolimus and mycophenolate mofetil, can improve the outcomes of ABOi transplantation by suppressing ABMR; however, this strong, nonspecific immunosuppression also increases infectious complications.³

Regulatory T cells (Tregs) can contribute to donor-specific transplantation tolerance while having much fewer adverse effects than nonspecific immunosuppression.⁴ Infusion of Tregs has been shown to suppress allograft rejection.⁴ However, the number of antigen-specific Tregs is very low and Tregs often lose their viability and activity after infusion. Recently, chimeric antigen receptor (CAR) T cells were developed and showed strong anti-tumor effects by specifically targeting tumor antigens.⁵ In parallel, CAR Tregs were developed to augment the antigen-specificity, viability as well as activity of conventional Tregs.⁶⁻⁸ A CAR consists of a single chain variable fragment (scFv) of an antibody in the extracellular domain to provide antigen-specificity to Tregs; and they also possess costimulatory molecules in the intracellular domains to improve the viability and activity of Tregs.

Complement activation is often involved in ABMR and the deposition of complement component 4d (C4d) as a byproduct of antibody-mediated complement activation is included in the diagnostic criteria of ABMR.⁹ Interestingly, C4d deposition is observed in 80-90% of ABOi transplantation cases as a result of either ABMR or accommodation, where antibody binding and subsequent activation of the proximal complement cascade occur without further tissue injury.¹⁰⁻¹²

To address the above problems in the art, described herein are compositions and methods that can effectively suppress ABMR and allograft rejection.

### BRIEF SUMMARY

Described herein are compositions and methods that can effectively suppress ABMR and allograft rejection. In one aspect, the composition comprises a genetically modified regulatory T cell (Treg), the Treg comprising an antigen binding protein that specifically binds complement component 4d (C4d). It will be understood that the genetically modified regulatory T cells described herein do not exist in nature.

In some embodiments, the antigen binding protein comprises a chimeric antigen receptor (CAR). In some embodiments, the CAR comprises a scFv that specifically binds C4d.

In some embodiments, the ABP, CAR or scFv comprises: a light chain variable region (VL) comprising:
a complementary determining region (CDR) 1 comprising an amino acid sequence SGSSGSYG, or a variant LCDR1 in which 1, 2, 3, 4, or 5 amino acids are substituted relative to the sequence;
a LCDR2 comprising an amino acid sequence YNDKRPS, or a variant LCDR2 in which 1, 2, 3, 4, or 5 amino acids are substituted relative to the sequence;
a LCDR3 comprising an amino acid sequence GSEDSSYVGV, or a variant LCDR3 in which 1, 2, 3, 4, or 5 amino acids are substituted relative to the sequence; and
a heavy chain variable region (VH) comprising:
a HCDR1 comprising an amino acid sequence SYALE, or a variant HCDR1 in which 1, 2, 3, 4, or 5 amino acids are substituted relative to the sequence;
a HCDR2 comprising an amino acid sequence GISSSGSGTNYGSAVKG, or a variant HCDR2 in which 1, 2, 3, 4, or 5 amino acids are substituted relative to the sequence;
a HCDR3 comprising an amino acid sequence AYGYVDAYGIDA, or a variant HCDR3 in which 1, 2, 3, 4, or 5 amino acids are substituted relative to the sequence.

In some embodiments, the VL comprises an amino acid sequence having at least 95% identity to

LTQPSSVSANPGGTVEITCSGSSGSYGWYQQKSPGSAPVTVIYYNDKRPSDIPSRFSGSKS GSTATLTITGVQAEDEAVYFCGSEDSSYVGVFGAGTTLTVL (SEQ ID NO: 2); and the VH comprises an amino acid sequence having at least 95% identity to

### AVTLDESGGGLQTPGGTLSLVCKGSGFTFRSYALEWVRQAPGKGLEYVAGISSSGSGTN YGSAVKGRATISRDNGQSTVRLQLNNLRAEDTGTYYCAKSAYGYVDAYGIDAWGHGT EVIVSSTS (SEQ ID NO: 4).

In some embodiments, the ABP, CAR or scFv comprises: a light chain variable region (VL) comprising:
a LCDR1 comprising an amino acid sequence SGGGRWYG, or a variant LCDR1 in which 1, 2, 3, 4, or 5 amino acids are substituted relative to the sequence;
a LCDR2 comprising an amino acid sequence HANTKRPS, or a variant LCDR2 in which 1, 2, 3, 4, or 5 amino acids are substituted relative to the sequence;
a LCDR3 comprising an amino acid sequence GSGDSSTDSGI, or a variant LCDR3 in which 1, 2, 3, 4, or 5 amino acids are substituted relative to the sequence; and
a heavy chain variable region (VH) comprising:
a HCDR1 comprising an amino acid sequence DRAIVIH, or a variant HCDR1 in which 1, 2, 3, 4, or 5 amino acids are substituted relative to the sequence;
a HCDR2 comprising an amino acid sequence GIYSSGRYTGYGSAVKG, or a variant HCDR2 in which 1, 2, 3, 4, or 5 amino acids are substituted relative to the sequence;
a HCDR3 comprising an amino acid sequence AGSIYCGYADVACIDA, or a variant HCDR3 in which 1, 2, 3, 4, or 5 amino acids are substituted relative to the sequence.

In some embodiments, the VL comprises an amino acid sequence having at least 95% identity to
LTQPSSVSANPGETVKITCSGGGRWYGWYQQKSPGSAPVTLIHANTKRPSNIPSRFSGSL SGSTSTLTISGVQAEDEAVYFCGSGDSSTDSGIFGAGTTLTVL (SEQ ID NO: 6); and the VH comprises an amino acid sequence having at least 95% identity to
AVTLDESGGGLQTPGGALSLVCKASGFSFSDRAMEIWVRQAPGKGLEWVAGIYSSGRYT GYGSAVKGRATISRDNGQSTVRLQLNNLRAEDTGTYYCAKAGSIYCGYADVACIDAW GHGTEVIVSSTS (SEQ ID NO: 8).

In some embodiments, the ABP, CAR or scFv comprises: a light chain variable region (VL) comprising:
a LCDR1 comprising an amino acid sequence SGGGSYYG, or a variant LCDR1 in which 1, 2, 3, 4, or 5 amino acids are substituted relative to the sequence;
a LCDR2 comprising an amino acid sequence SNNKRPS, or a variant LCDR2 in which 1, 2, 3, 4, or 5 amino acids are substituted relative to the sequence;
a LCDR3 comprising an amino acid sequence GSYDSNAGI, or a variant LCDR3 in which 1, 2, 3, 4, or 5 amino acids are substituted relative to the sequence; and
a heavy chain variable region (VH) comprising:
a HCDR1 comprising an amino acid sequence SYAMG, or a variant HCDR1 in which 1, 2, 3, 4, or 5 amino acids are substituted relative to the sequence;
a HCDR2 comprising an amino acid sequence EISGSGTSTYYGPAVKG, or a variant HCDR2 in which 1, 2, 3, 4, or 5 amino acids are substituted relative to the sequence;
a HCDR3 comprising an amino acid sequence CTRGGGAGSYIDA, or a variant HCDR3 in which 1, 2, 3, 4, or 5 amino acids are substituted relative to the sequence.

In any of the embodiments described herein, a CDR amino acid sequence can comprise one or more conservative amino acid substitutions relative to the recited sequence. For example, in any of the embodiments described herein, a CDR amino acid sequence can comprise 1, 2, 3, 4, or 5 conservative amino acid substitutions relative to the recited sequence. In some embodiments, the conservative amino acid substitutions do not substantially decrease binding affinity of the ABP, scFv or CAR to a target antigen such as mouse or human C4d.

In some embodiments, the VL comprises an amino acid sequence having at least 95% identity to

LTQPSSVSANPGETVEITCSGGGSYYGWYQQKSPGSAPVTVIYSNNKRPSDIPSRFSGSKS GSTSTLTITGVQADDEAVYYCGSYDSNAGIFGAGTTLTVL; and the VH comprises an amino acid sequence having at least 95% identity to

### AVTLDESGGGLQTPGGALSLVCKASGFTFSSYAMGWMRQAPGKGLDFVAEISGSGTST YYGPAVKGRATISRDNGRSTVRLQLNNLRAEDTGTYFCTRGGGAGSYIDAWGHGTEVI VSSTS.

In some embodiments, the ABP, CAR or scFv comprises a light chain amino acid sequence having at least 80% sequence identity to SEQ ID NO:2, or SEQ ID NO:6, or

### LTQPSSVSANPGETVEITCSGGGSYYGWYQQKSPGSAPVTVIYSNNKRPSDIPSRFSGSKS GSTSTLTITGVQADDEAVYYCGSYDSNAGIFGAGTTLTVL.

In some embodiments, the ABP, CAR or scFv comprises a heavy chain amino acid sequence having at least 80% sequence identity to SEQ ID NO:4, or SEQ ID NO:8, or

### AVTLDESGGGLQTPGGALSLVCKASGFTFSSYAMGWMRQAPGKGLDFVAEISGSGTST YYGPAVKGRATISRDNGRSTVRLQLNNLRAEDTGTYFCTRGGGAGSYIDAWGHGTEVI VSSTS.

In some embodiments, the scFv comprises an amino acid sequence having at least 80% sequence identity to:

i)
LTQPSSVSANPGGTVEITCSGSSGSYG WYQQKSPGSAPVTVIYYNDKRPS
DIPSRFSGSKSGSTATLTITGVQAEDEA VYFCGSEDSSYVGV FGAGTTLTVL
GQSSRSSGGGGSSGGGGS
AVTLDESGGGLQTPGGTLSLVCKGSGFTFRSYALE
WVRQAPGKGLEYVAGISSSGSGTNYGSAVKG
RATISRDNGQSTVRLQLNNLRAEDTGTYYCAKSAYGYVDAYGIDA WGHGTEVIVSSTS;
ii)
LTQPSSVSANPGETVKITCSGGGRWYG WYQQKSPGSAPVTLIHANTKRPS
NIPSRFSGSLSGSTSTLTISGVQAEDEAVYFCGSGDSSTDSGI FGAGTTLTVL
GQSSRSSGGGGSSGGGGS
AVTLDESGGGLQTPGGALSLVCKASGFSFSDRAMH
WVRQAPGKGLEWVAGIYSSGRYTGYGSAVKG
RATISRDNGQSTVRLQLNNLRAEDTGTYYCAKAGSIYCGYADVACIDA
WGHGTEVIVSST;
or
iii)
LTQPSSVSANPGETVEITCSGGGSYYGWYQQKSPGSAPVTVIYSNNKRPSDIPSRFSGSKS
GSTSTLTITGVQADDEAVYYCGSYDSNAGIFGAGTTLTVL
GQSSRSSGGGGSSGGGGS
AVTLDESGGGLQTPGGALSLVCKASGFTFSSYAMGWMRQAPGKGLDFVAEISGSGTST
YYGPAVKGRATISRDNGRSTVRLQLNNLRAEDTGTYFCTRGGGAGSYIDAWGHGTEVI
VSSTS.

In some embodiments, the CAR comprises a leader sequence. In some embodiments, the CAR comprises a hinge region, such as a human CD8 hinge region. In some embodiments, the hinge region comprises the amino acid sequence of SEQ ID NO: 10, or a sequence having at least 80% identity to SEQ ID NO: 10.

In some embodiments, the CAR comprises a CD28 transmembrane domain. In some embodiments, the CAR comprises a CD28 cytoplasmic domain. In some embodiments, the CAR comprises a CD28 transmembrane and cytoplasmic domain. In some embodiments, the CD28 transmembrane and cytoplasmic domain comprises the amino acid sequence of SEQ ID NO: 12, or a sequence having at least 80% identity to SEQ ID NO: 12.

In some embodiments, the CAR comprises a CD3 zeta cytoplasmic domain. In some embodiments, the CD3 zeta cytoplasmic domain comprises the amino acid sequence of SEQ ID NO: 14, or a sequence having at least 80% identity to SEQ ID NO: 14.

In some embodiments, the CAR comprises a c-myc tag.

In another aspect, provided is a regulatory T cell comprising a nucleic acid encoding an antigen binding protein, CAR or scFV described herein.

In another aspect, a vector is provided, the vector comprising a nucleic acid encoding an antigen binding protein, CAR or scFv described herein. In some embodiments, the vector is a retroviral vector.

In another aspect, a cell comprising a nucleic acid or vector described herein is provided. In some embodiments, the cell is a mammalian cell or cell line. In some embodiments, the cell is an immune cell, such as a T cell. In some embodiments, the cell is a regulatory T cell.

In another aspect, a method for producing regulatory T cells is described, the method comprising transfecting or transducing a regulatory T cell with a nucleic acid or vector comprising nucleic acid sequences encoding an antigen binding protein described herein, and selecting a Treg that expresses the antigen binding protein. In some embodiments, the antigen binding protein comprises a chimeric antigen receptor (CAR). In some embodiments, the CAR comprises a scFv that specifically binds C4d.

In another aspect, an in vitro method for inducing an immune response is described, the method comprising contacting a genetically modified regulatory T cell described herein with C4d antigen. In some embodiments, the step of contacting a genetically modified regulatory T cell described herein with C4d antigen results in expression of cell surface molecules and/or cytokines that suppress an immune response. In some embodiments, after contact with C4d antigen, the regulatory T cell upregulates CD69 expression and secretes increased levels IL-10 and IFN-γ compared to a control regulatory T cell that does not express an antigen binding protein that specifically binds C4d.

In another aspect, an in vitro method for suppressing T cell proliferation is described, the method comprising culturing a genetically modified regulatory T cell described herein with an activated effector T cell and determining a decrease in proliferation of the effector T cell.

In another aspect, a method for suppressing antibody-mediated rejection (ABMR) in a subject is described, the method comprising administering a therapeutically effective amount of a genetically modified regulatory T cell described herein to a subject. In some embodiments, the subject has previously received a transplant. In some embodiments, the subject is concurrently receiving a transplant. In some embodiments, the transplant is a allograft. In some embodiments, the allograft is an ABO blood group-incompatible (ABOi) allograft. In some embodiments, the allograft is a heart allograft.

### BRIEF DESCRIPTION OF THE DRAWINGS

FIG. 1A-1B. Production of anti-C4d CAR. FIG. 1A. Binding affinity of anti-C4d scFv clones (SC-8-Cκ, BF-2-Cκ) and control scFv clone (palivizumab-Cκ) to mouse C4d⁺Raji cells was measured by flow cytometric analysis. FIG. 1B. Structures of anti-C4d CAR, control CAR and anti-C4d CAR Tregs. CAR, chimeric antigen receptor; C4d, complement component 4d; Cyt, cytoplasmic domain; LS, leader sequence; mC4d, mouse complement component 4d; Myc, myc-tag; scFv, single chain variable fragment; TM, transmembraneous domain; Tregs, regulatory T cells; V_{H}, variable region of heavy chain; V_{L}, variable region of light chain.

FIGS. 2A, 2B, and 2C. Generation and phenotypes of anti-C4d CAR Tregs. FIG. 2A. Scheme of generation of anti-C4d CAR Tregs. Sorted CD62L⁺CD4⁺CD25⁺ Tregs were transduced with retrovirus containing either anti-C4d CAR or control CAR and then stimulated by anti-CD3/CD28 beads in the presence of IL-2 and rapamycin. FIG. 2B. Expression of Foxp3 and myc in CAR Tregs compared to NT Tregs along with viability (7-AAD) were measured by flow cytometry after the completion of generation on day 13. FIG. 2C. Expression of Foxp3, CD25, CTLA-4, LAP, and GITR in anti-C4d CAR Tregs compared to that in control CAR Tregs and NT Tregs. Abbreviations: CAR Tregs, chimeric antigen receptor regulatory T cells; CTLA-4, cytotoxic T-lymphocyte-associated protein 4; Foxp3, forkhead box P3; GITR, glucocorticoid-induced tumor necrosis factor receptor-related protein; IL-2, interleukin-2; LAP, latency-associated peptide; NT, non-transduced.

FIGS. 3A, 3B, and 3C. Specific binding to C4d and in vitro immunosuppressive activity of anti-C4d CAR Tregs. FIG. 3A. Specific binding of anti-C4d CAR Tregs to C4d. ** P<0.01 compared to anti-C4d CAR Tregs group (Student's t test). FIG. 3B. CD69 expression and secretion of IL-10 and IFN-γ by activation of anti-C4d CAR Tregs in response to binding to C4d on Raji cells. ** P<0.01 compared to anti-C4d CAR Tregs group (Student's t test). FIG. 3C. In vitro immunosuppressive activity of anti-C4d CAR Tregs against T cell proliferation. T cell proliferation was displayed by histogram of CTV-labelled T cells and calculated as the division index. ** P<0.01 compared to effector T cells alone, ##P<0.01 compared to NT Tregs group (Student's t test). N=3 per each group. Each value in the bar charts indicates the mean and standard error of the mean. CAR Tregs, chimeric antigen receptor regulatory T cells; CTV, Cell Trace Violet; IFN-γ, interferon-γ; IL-10, interleukin-10; MFI, mean fluorescence intensity; NT, non-transduced; Tresp, responder T cells.

FIGS. 4A, 4B, 4C, and 4D. Immunosuppressive activity of anti-C4d CAR Tregs against allograft rejection in ABOi heart transplantation. FIG. 4A. Overall scheme of ABOi heart transplantation and immunosuppressive regimens. Sensitized C57BL6/J mice underwent A-TG BALB/c heart transplantation one day after adoptive transfer of anti-C4d CAR Tregs, control CAR Tregs or NT Tregs. Serum titers of anti-A IgM and IgG were measured by flow cytometric analysis. FIG. 4B. Heart allograft survival rates in ABOi heart transplantation. *P<0.01 compared to PBS group; P<0.01 compared to control CAR Treg group (Log rank test). FIG. 4C. Expression of proinflammatory cytokines (IL-1β, IL-6, and IFN-γ) in heart allograft was measured by real time PCR. Each value in bar charts indicates the mean and standard error of the mean. ** P<0.01 compared to PBS control (Student's t test). FIG. 4D. H&E staining imaging (magnification×200) to show ABOi allograft injury and merged views in IF imaging (magnification×400) to show infiltration of CD45.1⁺Myc⁺ anti-C4d CAR Tregs into C4d⁺ ABOi heart allograft tissues (C4d, green; CD45.1, red; Myc, yellow; blue, DAPI). ABOi, ABO-incompatible; A-TG, human blood group A antigen-transgenic; CAR Tregs, chimeric antigen receptor regulatory T cells; DAPI, 4,6-diamidino-2-phenylindole; H&E, Hematoxylin and eosin; IF, Immunofluorescence staining; IL, interleukin; IFN-γ, interferon-γ; NT, non-transduced; PCR, polymerase chain reaction; WT, wild-type.

FIG. 5A shows a representative protocol for expansion of regulatory T cells by polyclonal stimulation in cultures comprising L-cells. FIG. 5B shows a survival curve comparing anti-C4d CAR Treg cells to control cells. The data were generated as described in FIG. 4A-4D.

FIGS. 6A and 6B show a representative protocol for generating anti-human C4d CAR regulatory T cells (anti-human C4d CAR-Treg). FIG. 6A shows a representative gating strategy to isolate human regulatory T cells. FIG. 6B shows representative markers expressed by regulatory T cells transduced with control and anti-human C4d CAR.

FIG. 7 shows a representative protocol for generating anti-human C4d CAR regulatory T cells (anti-human C4d CAR-Treg) by polyclonal stimulation in cultures comprising K562 cells.

### NOMENCLATURE

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “or” should be understood to mean either one, both, or any combination thereof of the recited alternatives. Recitation of ranges of values herein are merely intended to serve as a shorthand method of referring individually to each separate value falling within the range, including the end points of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

As used herein, the term “regulatory T cell” (“Treg”) refers to a T cell which regulates or suppresses the function of other cells in the immune system. For example, Tregs suppress activation, proliferation and cytokine production of CD4+ T cells and CD8+ T cells, and may also suppress B cells and dendritic cells. Tregs can produce soluble messengers which have a suppressive function, including TGF-beta, IL-10 and adenosine. Tregs typically express the cell surface markers CD4 (T cell co-receptor) and CD25, and also the nuclear transcription factor Forkhead box P3 (FoxP3). See the internet at immunology.org/public-information/bitesized-immunology/cells/regulatory-t-cells-tregs.

As used herein, the term “antigen binding protein” (ABP) refers to a protein that specifically binds a target antigen, and includes antibodies, scFv's and CARs described herein. The term “antigen binding domain” refers to the portion of an antigen binding protein that specifically binds to a target antigen.

As used herein, the term “antibody” refers to an immunoglobulin (Ig) molecule or format fragment thereof that specifically binds to a target antigen. The term includes monoclonal antibodies and the IgA, IgD, IgE, IgG, and IgM isotypes and subtypes. The term also includes antigen-binding fragments or formats thereof, such as Fab (fragment, antigen binding), Fv (variable domain), scFv (single chain fragment variable), disulfide-bond stabilized scFv (ds-scFv), single chain Fab (scFab), dimeric and multimeric antibody formats like dia-, tria- and tetra-bodies, minibodies (miniAbs) comprising scFvs linked to oligomerization domains, VHH/VH of camelid heavy chain Abs and single domain Abs (sdAb). The term also includes fusion proteins of antibodies or antigen-binding fragments thereof, such as scFv-light chain fusion proteins, or scFv-Fc fusion proteins. The term also includes antibodies or antigen-binding fragments thereof that include an Fc domain to provide effector functions such as Antibody-Dependent Cell-Mediated Cytotoxicity (ADCC) and Complement Dependent Cytotoxicity (CDC).

The term “humanized” refers to an antigen binding protein, or antigen binding fragments or formats thereof, from a non-human species that is modified to include human amino acid sequences. The humanized ABP can include sequences that reduce potential immunogenicity when the ABP is administered to a human. For example, the humanized ABP can include the complementarity-determining regions (CDRs) from a non-human species and the antibody framework or scaffold regions from a human antibody.

As used herein, the term “specifically binds” refers to the strength or binding affinity between an antigen binding protein and its cognate target antigen as compared to binding by a control or non-specific antigen binding protein. Affinity refers to the strength of binding of a single molecule to its ligand and is typically determined by the equilibrium dissociation constant (K_{D}). K_{D} is the ratio of the dissociation rate (koff) (how quickly the ABP dissociates from its antigen), to the association rate (kon) (how quickly the ABP binds to its antigen). KD values can be determined by measuring the kon and koff rates of a specific ABP or antibody/antigen interaction and then using a ratio of these values to calculate the KD value. An antibody that specifically binds a target antigen typically has a K_{D} value in the low micromolar (10-6) to picomolar (10⁻¹²) range. High affinity antibodies generally have a K_{D} in the low nanomolar range (10-9), whereas very high affinity antibodies can have a K_{D} in the picomolar (10⁻¹²) range.

As used herein, the term “substantially identical,” in reference to a nucleic acid or amino acid sequence, refers to two sequences that have at least about 30% to at least about 99.9% sequence identity, (for example, at least about 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity) over a specified region when compared and aligned for maximum correspondence over a comparison window, or designated region as measured using a sequence comparison algorithm, or by manual alignment and visual inspection. The term “substantially identical” also includes sequences that are less than 100% identical, for example, sequences that have 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. In some embodiments, two proteins (or a region of the proteins) are substantially identical when the amino acid sequences have at least about 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. In some embodiments, two proteins (or a region of the proteins) are substantially identical when the amino acid sequences have about 30%, 40%, 50% 60%, 65%, 70%, 75%, 80%, 85%, 90%, 91%, 92%, 93%, 94%, 95%, 96%, 97%, 98%, 99%, 99.5% or 99.9% sequence identity. In some embodiments, two proteins (or a region of the proteins) are substantially identical when the amino acid sequences have greater than 30% identity but less than 100% identity, for example 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. In some embodiments, two nucleic acid sequences are substantially identical when the nucleic acid sequences have at least about 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. In some embodiments, two nucleic acid sequences are substantially identical when the nucleic acid sequences have about 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. In some embodiments, two nucleic acid sequences are substantially identical when the nucleic acid sequences have greater than 30% identity but less than 100% identity, for example 30%, 40%, 50% 60%, 65%, 70%, 75%, 80%, 85%, 90%, 91%, 92%, 93%, 94%, 95%, 96%, 97%, 98%, 99%, 99.5%, or 99.9% sequence identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, at least 40%, at least 50%, at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. The BLAST computer program can be used to align and determine the percent sequence identity between nucleic acid and amino acid sequences.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

The term “subject” refers to an animal, for example a mammal, including but not limited to a human, a rodent such as a mouse or rat, a companion animal such as a dog or cat, and livestock such as cows, horses, and sheep. The term subject can also be used interchangeably with the term “patient.”

As used herein, the term “operably linked” refers to a functional linkage between nucleic acid promoter and/or regulatory sequences and protein coding sequences, such that the linked promoter and/or regulatory sequences functionally controls expression of the protein coding sequence.

The term “about,” when modifying a numerical value or range of values described herein, includes values encompassing normal variation and experimental error in the art, and can include +/− 10% of the recited value or range, such as +/−1%, 2%, 3%, 4%, 5%, 6%, 7%, 8%, 9% or 10% of the recited value or range.

The term “complementarity-determining region” (CDR) refers to amino acid sequence located within the variable regions or domains of antibodies that bind a specific antigen. CDRs are described by Kabat et al., J. Biol. Chem. 252:6609-6616 (1977); Kabat et al., U.S. Dept. of Health and Human Services, “Sequences of proteins of immunological interest” (1991); by Al-Lazikani, B.; Lesk, A. M.; Chothia, C. (1997). “Standard conformations for the canonical structures of immunoglobulins”. Journal of Molecular Biology. 273 (4): 927-948; and North, B.; Lehmann, A.; Dunbrack Jr, R. L. (2011). “A New Clustering of Antibody CDR Loop Conformations”. Journal of Molecular Biology. 406 (2): 228-256. As is well understood in the art, there a three non-contiguous CDRS (CDR1, CDR2 and CDR3) in the amino acid sequence of each variable region, i.e., the light chain variable region (VL) and the heavy chain variable region (VH). The CDRs in the VL are referred to as LCDR1, LCDR2, and LCDR3. The CDRs in the VH are referred to as HCDR1, HCDR2, and HCDR3.

The tern “genetically modified regulatory T cell” refers to a Treg cell that has been transfected or transduced with an exogenous nucleic acid sequence or a vector comprising an exogenous nucleic acid sequence. The term includes a Treg cell expressing an anti-C4d ABP, anti-C4d scFv or anti-C4d CAR described herein.

### DETAILED DESCRIPTION

Described herein are compositions that are useful for suppressing ABMR and allograft rejection, and methods of making and using the compositions. In one aspect, the compositions comprise genetically modified regulatory T cells (Tregs) that express an antigen binding protein that specifically binds complement component 4d (C4d) (anti-C4d Tregs). The genetically modified anti-C4d Tregs provide the following unexpected advantages. First, they are effective at suppressing ABMR and allograft rejection when administered to a subject. Second, they are effective at suppressing in vitro T cell proliferation as well as proliferation of non-transduced Tregs. Third, adoptive transfer of the anti-C4d Tregs significantly prolonged heart allograft survival in a mouse model. The anti-C4d Tregs therefore represent promising therapeutic agents for controlling ABMR, including rejection associated with ABOi allografts.

### Regulatory T Cells (Tregs) That Specifically Bind Complement Component 4d (C4d)

In one aspect, described herein are genetically modified Tregs that can specifically bind complement component 4d (C4d). In some embodiments, the modified Tregs express an antigen binding protein (ABP) that specifically binds C4d, or an antigenic fragment thereof. In some embodiments, the modified Tregs express an antigen binding protein (ABP) that specifically binds a mammalian C4d, or an antigenic fragment thereof. In some embodiments, the modified Tregs express an antigen binding protein (ABP) that specifically binds a rodent (e.g., rat or mouse) C4d, or an antigenic fragment thereof. In some embodiments, the modified Tregs express an antigen binding protein (ABP) that specifically binds human C4d, or an antigenic fragment thereof. In any of the embodiments described herein, the modified Tregs express an antigen binding protein (ABP) that specifically binds a C4d-Fc fusion protein, such as a C4d-human Fc fusion protein.

In some embodiments, the ABP is an antibody or antigen-binding format thereof. In some embodiments, the ABP is a chimeric or humanized antibody or antigen-binding fragment or format thereof In some embodiments, the ABP is a single-chain variable fragment (scFv). In some embodiments, the ABP is a chimeric antigen receptor (CAR).

In some embodiments, the CAR comprises one or more of the following elements: (i) an antibody that binds C4d (an anti-C4d antibody); (ii) a hinge region; (iii) a transmembrane domain; (iv) a cytoplasmic domain; and/or (v) a leader sequence, or combinations thereof. In some embodiments, the CAR comprises elements in the following order starting at the amino terminus: (i) a leader sequence; (ii) an anti-C4d antibody; (iii) a hinge region; (iv) a transmembrane domain; and (v) a cytoplasmic domain. The cytoplasmic domain contains amino acid sequences that are responsible for T cell activation.

In any of the embodiments described herein, the anti-C4d antibody is an scFv that specifically binds C4d. In some embodiments, the scFv comprises heavy and/or light chain sequences from a mammal, such as a human or mouse, or from an Ayes species, such as a chicken. In some embodiments, the anti-C4d antibody or anti-C4d scFv comprises chimeric or humanized sequences. In some embodiments, the anti-C4d antibody comprises a chimeric or humanized C4d antibody, or antigen binding fragment or format thereof. In some embodiments, 10 the light chain variable region of the anti-C4d antibody or scFv comprises an amino acid sequence that is substantially identical to SEQ ID NO:2 or SEQ ID NO:6. In some embodiments, the light chain variable region of the anti-C4d antibody or scFv comprises SEQ ID NO:2 or SEQ ID NO:6. In some embodiments, the heavy chain variable region of the anti-C4d antibody or scFv comprises an amino acid sequence that is substantially identical to SEQ ID NO:4 or SEQ ID NO:8. In some embodiments, the heavy chain variable region of the anti-C4d antibody or scFv comprises SEQ ID NO:4 or SEQ ID NO:8.

In some embodiments, the anti-C4d antibody or anti-C4d scFv binds a mammalian C4d, including but not limited to a rodent (e.g., rat or mouse) or human C4d, or an antigenic fragment thereof. In some embodiments, the anti-C4d antibody or anti-C4d scFv binds a C4d-Fc fusion protein.

In some embodiments, the hinge region is a human CD8 hinge region. In some embodiments, the hinge region comprises an amino acid sequence that is substantially identical to SEQ ID NO:10. In some embodiments, the hinge region comprises SEQ ID NO:10.

In some embodiments, the transmembrane domain comprises a CD28 transmembrane domain. In some embodiments, the transmembrane domain comprises a mouse CD28 transmembrane domain.

In some embodiments, the CAR comprises a cytoplasmic domain comprising a costimulatory domain selected from CD28, CD27, 4-1BB, or OX40. In some embodiments, the cytoplasmic domain comprises a CD28 costimulatory-signaling domain. In some embodiments, the cytoplasmic domain comprises a mouse CD28 costimulatory-signaling domain.

In some embodiments, the CAR comprises a CD28 transmembrane and/or a CD28 cytoplasmic domain. In some embodiments, the CAR comprises a mouse CD28 transmembrane and/or a mouse CD28 cytoplasmic domains. In some embodiments, the CD28 transmembrane and cytoplasmic domains comprise an amino acid sequence that is substantially identical to SEQ ID NO:12. In some embodiments, the CD28 transmembrane and cytoplasmic domains comprise SEQ ID NO:12.

In some embodiments, the cytoplasmic domain comprises a CD3 (CD3zeta) cytoplasmic domain. In some embodiments, the cytoplasmic domain comprises a human CD3ζ cytoplasmic domain. In some embodiments, the human CD3ζ cytoplasmic domain comprises an amino acid sequence that is substantially identical to SEQ ID NO:22. In some embodiments, the human CD3ζ cytoplasmic domain comprises SEQ ID NO:22. In some embodiments, the cytoplasmic domain comprises a mouse CD3ζ cytoplasmic domain. In some embodiments, the mouse CD3ζ cytoplasmic domain comprises an amino acid sequence that is substantially identical to SEQ ID NO:14. In some embodiments, the mouse CD3ζ cytoplasmic domain comprises SEQ ID NO:14.

In some embodiments, the cytoplasmic domain comprises one or more Immune-receptor-Tyrosine-based-Activation-Motif (ITAM) sequences.

In some embodiments, the cytoplasmic domain comprises a fusion protein comprising a CD28 costimulatory-signaling domain and a CD3ζ cytoplasmic domain. In some embodiments, the cytoplasmic domain comprises a fusion protein comprising a mouse CD28 costimulatory-signaling domain and a human CD3ζ cytoplasmic domain. In some embodiments, the cytoplasmic domain comprises a fusion protein comprising a mouse CD28 costimulatory-signaling domain and a mouse CD3ζ cytoplasmic domain.

In some embodiments, the CAR comprises a CD28 extracellular domain, a CD28 transmembrane domain, and/or a CD28 cytoplasmic domain, or combinations thereof. In some embodiments, the CAR comprises a mouse CD28 extracellular domain, a mouse CD28 transmembrane domain, and/or a mouse CD28 cytoplasmic domain, or combinations thereof. In some embodiments, the CAR comprises a mouse CD28 extracellular domain, a mouse CD28 transmembrane domain, and a mouse CD28 cytoplasmic domain having an amino acid sequence that is substantially identical to SEQ ID NO: 20. In some embodiments, the CAR comprises a CD28 extracellular domain, a CD28 transmembrane domain, and a CD28 cytoplasmic domain having an amino acid sequence comprising SEQ ID NO: 20.

In some embodiments, the CAR comprises a fusion protein comprising a CD28 extracellular domain, a CD28 transmembrane domain, a CD28 cytoplasmic domain and a CD3-zeta cytoplasmic domain. In some embodiments, the CAR comprises a fusion protein comprising a mouse CD28 extracellular domain, a mouse CD28 transmembrane domain, a mouse CD28 cytoplasmic domain and/or a mouse CD3-zeta cytoplasmic domain, or combinations thereof. In some embodiments, the CAR comprises a fusion protein comprising a mouse CD28 extracellular domain, a mouse CD28 transmembrane domain, a mouse CD28 cytoplasmic domain and a mouse CD3-zeta cytoplasmic domain having an amino acid sequence that is substantially identical to SEQ ID NO: 24. In some embodiments, the CAR comprises a fusion protein comprising a CD28 extracellular domain, a CD28 transmembrane domain, a CD28 cytoplasmic domain and a CD3-zeta cytoplasmic having an amino acid sequence comprising SEQ ID NO: 24.

In some embodiments, the CAR further comprises an amino acid tag, such as a c-myc tag that is useful for sorting and selecting cells transfected with the CAR. In some embodiments, the tag is located between the anti-C4d antibody and the hinge region of the CAR. In some embodiments, the tag comprises an amino acid sequence that is substantially identical to SEQ ID NO:26. In some embodiments, the tag comprises the amino acid sequence of SEQ ID NO:26.

In some embodiments, the CAR comprises an amino acid sequence that is substantially identical to SEQ ID NO:16 or SEQ ID NO:18. In some embodiments, the CAR comprises the amino acid sequence of SEQ ID NO:16 or SEQ ID NO:18.

In some embodiments, the genetically modified regulatory T cell expresses the markers CD62L, CD4, and CD25. In some embodiments, the genetically modified regulatory T cell expresses an anti-C4d ABP or CAR and also expresses Foxp3, CD25, CTLA-4, LAP, and GITR at similar levels as a control (e.g., non-transduced) or natural regulatory T cell.

### Nucleic Acids

Also described are nucleic acid molecules and/or nucleic acid sequences that encode one or more components of the antigen binding proteins described herein. In some embodiments, the nucleic acid molecules comprise a nucleic acid sequence encoding one or more components of a CAR described herein. For example, in some embodiments, the nucleic acid molecules comprise a sequence encoding (i) a leader sequence; (ii) an anti-C4d antibody; (iii) a hinge region; (iv) a transmembrane domain; and/or (v) a cytoplasmic domain.

In some embodiments, the nucleic acid sequence encodes an anti-C4d antibody. In some embodiments, the nucleic acid sequence encodes an anti-C4d scFv that specifically binds C4d. In some embodiments, the nucleic acid sequence encodes a light chain variable region of an anti-C4d antibody or scFv. In some embodiments, the nuclei acid molecule encoding the light chain variable region of the anti-C4d antibody or scFv comprises a nucleic acid sequence that is substantially identical to SEQ ID NO:1 or SEQ ID NO:5. In some embodiments, the nuclei acid molecule encoding the light chain variable region of the anti-C4d antibody or scFv comprises the nucleic acid sequence of SEQ ID NO:1 or SEQ ID NO:5.

In some embodiments, the nucleic acid sequence encodes a heavy chain variable region of the anti-C4d antibody or scFv. In some embodiments, the nuclei acid molecule encoding the heavy chain variable region of the anti-C4d antibody or scFv comprises a nucleic acid sequence that is substantially identical to SEQ ID NO:3 or SEQ ID NO:7. In some embodiments, the nuclei acid molecule encoding the heavy chain variable region of the anti-C4d antibody or scFv comprises the nucleic acid sequence of SEQ ID NO:3 or SEQ ID NO:7.

In some embodiments, the nucleic acid sequence encodes a hinge region. In some embodiments, the nucleic acid sequence encodes a human CD8 hinge region. In some embodiments, the nucleic acid molecule encoding the hinge region comprises a nucleic acid sequence that is substantially identical to SEQ ID NO:9. In some embodiments, the nucleic acid molecule encoding the hinge region comprises the nucleic acid sequence of SEQ ID NO:9.

In some embodiments, the nucleic acid sequence encodes a transmembrane domain. In some embodiments, the nucleic acid sequence encodes a CD28 transmembrane domain. In some embodiments, the nucleic acid sequence encodes a CD28 transmembrane and cytoplasmic domain. In some embodiments, the nucleic acid sequence encodes a mouse CD28 transmembrane and cytoplasmic domain. In some embodiments, the nucleic acid molecule encoding the transmembrane and cytoplasmic domain comprises a nucleic acid sequence that is substantially identical to SEQ ID NO:11. In some embodiments, the nucleic acid molecule encoding the transmembrane domain comprises the nucleic acid sequence of SEQ ID NO:11.

In some embodiments, the nucleic acid sequence encodes a CD28 extracellular domain, a CD28 transmembrane domain, and/or a CD28 cytoplasmic domain, or combinations thereof. In some embodiments, the nucleic acid sequence encodes a mouse CD28 extracellular domain, a mouse CD28 transmembrane domain, and/or a mouse CD28 cytoplasmic domain, or combinations thereof. In some embodiments, the nucleic acid sequence encoding the mouse CD28 extracellular domain, mouse CD28 transmembrane domain, and mouse CD28 cytoplasmic domain comprises a nucleic acid sequence that is substantially identical to SEQ ID NO:19. In some embodiments, the nucleic acid sequence encoding the mouse CD28 extracellular domain, mouse CD28 transmembrane domain, and mouse CD28 cytoplasmic domain comprises SEQ ID NO:19.

In some embodiments, the nucleic acid sequence encodes a CD3ζ (CD3zeta) cytoplasmic domain. In some embodiments, nucleic acid sequence encodes a human CD3ζ cytoplasmic domain. In some embodiments, the nucleic acid sequence encoding the human CD3ζ cytoplasmic domain comprises a nucleic acid sequence that is substantially identical to SEQ ID NO:21. In some embodiments, the nucleic acid sequence encoding the human CD3ζ cytoplasmic domain comprises SEQ ID NO:21. In some embodiments, the nucleic acid sequence encodes a mouse CD3ζ cytoplasmic domain. In some embodiments, the nucleic acid sequence encoding the mouse CD3ζ cytoplasmic domain comprises a nucleic acid sequence that is substantially identical to SEQ ID NO:14. In some embodiments, the nucleic acid sequence encoding the mouse CD3ζ cytoplasmic domain comprises SEQ ID NO:14.

In some embodiments, the nucleic acid sequence encodes a fusion protein comprising a CD28 extracellular domain, a CD28 transmembrane domain, a CD28 cytoplasmic domain and a CD3-zeta cytoplasmic domain. In some embodiments, the nucleic acid sequence encodes a fusion protein comprising a mouse CD28 extracellular domain, a mouse CD28 transmembrane domain, a mouse CD28 cytoplasmic domain and/or a mouse CD3-zeta cytoplasmic domain. In some embodiments, the nucleic acid encoding a fusion protein comprising a mouse CD28 extracellular domain, a mouse CD28 transmembrane domain, a mouse CD28 cytoplasmic domain and a mouse CD3-zeta cytoplasmic domain comprises a nucleic acid sequence that is substantially identical to SEQ ID NO: 23. In some embodiments, the nucleic acid encoding a fusion protein comprising a CD28 extracellular domain, a CD28 transmembrane domain, a CD28 cytoplasmic domain and a CD3-zeta cytoplasmic comprises the nucleic acid sequence of SEQ ID NO: 23.

In some embodiments, the nucleic acid encodes an amino acid tag, such as a c-myc tag that is useful for sorting and selecting cells transfected with the CAR. In some embodiments, the nucleic acid sequence encoding the tag is located between the nucleic acid sequences encoding the anti-C4d antibody/scFv and nucleic acid sequences encoding the hinge region of the CAR. In some embodiments, the nucleic acid encoding the tag comprises a nucleic acid sequence that is substantially identical to SEQ ID NO:25. In some embodiments, the nucleic acid encoding the tag comprises the nucleic acid sequence of SEQ ID NO:25.

In some embodiments, the nucleic acid sequence encoding the CAR comprises a nucleic acid sequence that is substantially identical to SEQ ID NO:15 or SEQ ID NO:17. In some embodiments, the nucleic acid sequence encoding the CAR comprises the nucleic acid sequence of SEQ ID NO:15 or SEQ ID NO:17.

In any of the embodiments described herein, the nucleic acid or amino acid sequence can comprise or consist of the recited SEQ ID NO.

### Vectors

In some aspects, the disclosure provides vectors comprising a nucleic acid sequence described herein. Vectors can be self-replicating in a host cell, or can be integrated into the genome of a host cell. In some embodiments, the vector is retroviral vector. In some embodiments, vector comprises a nucleic acid sequence encoding an scFv that binds C4d. In some embodiments, vector comprises a nucleic acid sequence encoding an scFv-Cκ fusion protein.

In some embodiments, the vector comprises one or more nucleic acid sequences encoding one or more elements of an anti-C4d CAR described herein. In some embodiments, the vector comprises one or more nucleic acid sequences encoding an anti-C4d antibody, an amino acid tag, a hinge region, a transmembrane region and/or a cytoplasmic region. In some embodiments, the vector comprises one or more nucleic acid sequences encoding the scFv format of an anti-C4d antibody, a c-myc tag, a human CD8 hinge region, a mouse CD28 transmembrane region and cytoplasmic region, and a human CD3ζ cytoplasmic region.

In some embodiments, the vector is an expression vector that comprises transcriptional and/or translational regulatory elements that regulate RNA and/or protein expression of nucleic acid sequences that are operably linked to the transcriptional and/or translational regulatory elements. In some embodiments, the vector comprises a promoter sequence operably linked to a nucleic acid sequence described herein. In some embodiments, promoter is a constitutive promoter. In some embodiments, promoter is an inducible promoter.

### Methods for Producing Modified Regulatory T Cells

In another aspect, methods for producing modified Tregs described herein are provided. In some embodiments, the methods comprise transfecting or transducing the regulatory T cell with a nucleic acid or vector described herein, where the nucleic acid or vector comprises a nucleic acid sequence encoding an anti-C4d ABP described herein. In some embodiments, the nucleic acid or vector comprises a nucleic acid sequence encoding an anti-C4d scFv described herein. After transfection or transduction, Tregs can be cultured under conditions that permit expression of antigen binding proteins encoded by the nucleic acid sequence. Tregs that express the anti-C4d ABP can then be selected, for example, by staining the Tregs with antibodies that bind to a component of the anti-C4d ABP and sorting the cells by flow cytometry or fluorescence-activated cell sorting (FACS). In some embodiments, Tregs that express the anti-C4d ABP can be selected by detecting the expression of an amino acid tag, such as a c-myc tag.

In some embodiments, modified Tregs are cultured with anti-CD3 and anti-CD28 antibodies and the cytokine IL-2. In some embodiments, modified Tregs are cultured with anti-CD3 and anti-CD28 antibodies, IL-2, and rapamycin.

### Methods for Inducing an Immune Response

In another aspect, methods for inducing an immune response are described. In some embodiments, the method comprises contacting a regulatory T cell expressing an anti-C4d ABP with C4d antigen, and determining if an immune response is produced. In some embodiments, the method is an in vitro method, and comprises culturing a modified regulatory T cell expressing an anti-C4d ABP with C4d antigen. In some embodiments, the C4d antigen is a soluble C4d antigen. In some embodiments, the C4d antigen is a soluble C4d antigen-human Fc fusion protein.

In some embodiments, the immune response comprises increased (upregulated) CD69 expression, increased IL-10 expression or secretion, and/or increased IFN-γ expression or secretion by the modified regulatory T cell compared to a control Treg that does not express an anti-C4d ABP (for example, a non-transduced Treg (NT Treg) or a Treg that expresses an irrelevant ABP, such as a control CAR containing a palivizumab scFv.

### Methods for Suppressing T Cell Proliferation

In another aspect, in vitro methods for suppressing T cell proliferation are provided. In some embodiments, the method comprises culturing a modified Treg expressing an anti-C4d ABP with activated effector T cells (Teff), and determining a decrease in proliferation and/or activation of the effector T cells. In some embodiments, the effector T cells are labeled with a fluorescent tracking dye, such as carboxyfluorescein succinimidyl ester (CF SE), before starting the experiment and by monitoring dilution of the dye in daughter cells as cells get activated and divide over time. In some embodiments, the modified Tregs are labeled with a different fluorescent tracking dye, such as CellTrace Violet dye (CTV), to exclude the Treg cells from the target Teff gate and monitor concurrent changes in Tregs from the same co-cultures. T cell suppression assays are described in Zappasodi, R. et al. “In vitro assays for effector T cell functions and activity of immunomodulatory antibodies.” Methods in enzymology vol. 631 (2020): 43-59. doi:10.1016/bs.mie.2019.08.012.

### Methods for Suppressing Antibody-mediated Rejection (ABMR)

In another aspect, in vivo methods for suppressing antibody-mediated rejection are provided. In some embodiments, the method comprises administering a regulatory T cell expressing an anti-C4d ABP to a subject having an organ transplant in an amount effective to decrease or prevent rejection of the transplanted organ. In some embodiments, the effective amount comprises administering about 1×10⁶ to about 1×10⁹ anti-C4d CAR expressing Treg cells to the subject. In some embodiments, the effective amount comprises administering about 1×10⁶/kg to about 1×10⁹/kg (body weight) anti-C4d CAR expressing Treg cells to the subject.

In some embodiments, the transplant is a allograft. In some embodiments, the allograft is an ABO blood group-incompatible (ABOi) allograft. In some embodiments, the allograft is a heart allograft.

In some embodiments, the subject is a mammal, such as but not limited to a rodent (e.g., a mouse or rat), cow, sheep, horse, pig, dog, cat, or non-human primate. In some embodiments, the subject is a human.

### EXAMPLES

### Example 1

### Materials & Methods

### Animals

C57BL/6J and CD45.1+ congenic C57BL/6J mice were purchased from Jackson Laboratory (Bar Harbor, Me., USA). Human blood group A antigen-transgenic (A-TG) BALB/c mice were generously provided by Peter Cowan ('St Vincent's Hospital, University of Melbourne, Australia) and Lori West (University of Alberta, Canada).13

### Generation of Combinatorial scFv-displayed Phage Library and Bio-panning

Four white leghorn chickens were immunized with 5 μg of recombinant mouse C4d-Fc using three boosters. After immunization, cDNA was synthesized, as previously described.¹⁴ Four rounds of bio-panning with C4d-Cκ-conjugated magnetic beads was performed.¹⁵ From the output titer plate, scFv clones were chosen randomly for the phage enzyme immunoassay using C4d-Cκ-coated microtiter plates (3690; Corning Life Sciences, Corning, NY, USA).¹⁶ Clones reactive (A405>2.0) to C4d-Cκ were sequenced using OmpSeq primers by Cosmogenetech (Seoul, Korea).17

### Expression and Purification of scFv-Cκ Fusion Protein

A pCEP4 expression vector was constructed to encode the scFv-Cκ fusion protein. Palivizumab scFv was also cloned into the vector as a control.¹⁵ The constructs were transfected into human embryonic kidney-293F cells (Invitrogen, Carlsbad, CA, USA) using FectoPRO (Polyplus, Illkirch, France) and purified by affinity chromatography as previously described.¹⁸

### Construction and Transfection of a Retroviral Vector

To construct a retroviral vector, a plasmid containing genes of the scFv fragment of an anti-C4d antibody, c-myc tag, human CD8 hinge region, mouse CD28 transmembrane region and cytoplasmic region, as well as a human CD3ζ cytoplasmic region, was synthesized by Integrated DNA Technologies (Coralville, Iowa, USA). The CAR construct was cloned downstream of the PGK promoter of the pMSCV-puro retroviral vector (Takara Bio, Shiga Japan). The detailed transfection and retrovirus packing procedures are described in A.1. Supplemental Methods.

### Generation of CAR Tregs

Sorted CD62L+CD4+CD25+T cells were stimulated with DYNABEADS™ Mouse T-

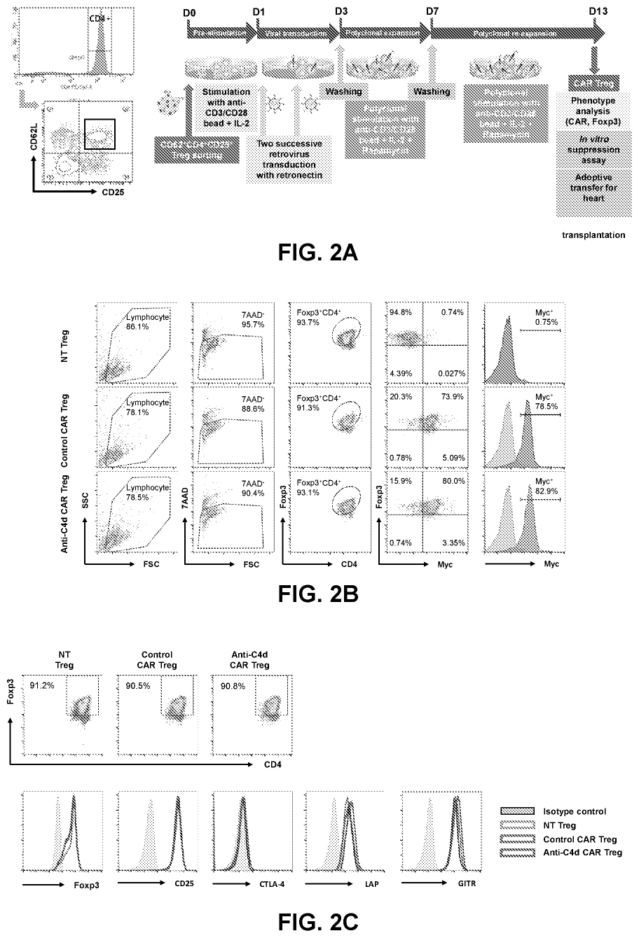

Activator CD3/CD28 (Thermo Fisher Scientific, Waltham, MA, USA) and interleukin (IL)-2 (4,000 IU, PROLEUKIN, Boehringer Ingelheim Pharma, Biberach/Riss, Germany) for one day (FIG. 2A). Next, these cells were transduced with retrovirus using retronection (Takara Bio) on two consecutive days. Transduced Tregs were stimulated by anti-CD3/CD28 Dynabeads in the presence of IL-2 and rapamycin (100 nM, Sigma-Aldrich, St Louis, MO, USA) for two rounds. As a control, non-transduced Tregs (NT Tregs) were stimulated in the same manner except viral transduction.

### Binding and Activation Assay for CAR Tregs

To assess binding of CAR Tregs to C4d, NT Tregs, control CAR Tregs, or anti-C4d CAR Tregs were cultured with C4d-human Fc for 2 h, and then anti-human Fc was added for flow cytometric analysis. For the activation assay, C4d+ Raji cells were co-cultured for 48 h with three groups of Tregs.¹⁹ Expression of CD69 in Tregs and secretion of IL-10 and interferon-γ (IFN-γ) were measured by flow cytometry and enzyme-linked immunosorbent assay (Biolegend, San Diego, CA, USA), respectively.

### In Vitro Suppression Assay

Splenic CD45.1+CD4+ T cells labeled with CTV (CELLTRACE™ Violet Cell Proliferation Kit, Thermo Fisher Scientific) were stimulated with anti-CD3/CD28 Dynabeads for 3 days with or without CD45.2+CAR Tregs at a ratio of 4 to 1. T cell proliferation was expressed as the division index.²⁰

### Heart Transplantation and Immunosuppressive Regimens

Wild-type C57BL/6J mice were sensitized by human blood group A antigen as previously described.²¹ Hearts from A-TG BALB/c mice were transplanted into the sensitized C57BL/6J mice. CD45.1+NT, control CAR, or anti-C4d CAR Tregs (1×106) were transferred into recipient mice one day before transplantation. Prednisolone (Yuhan, Seoul, Korea), tacrolimus (Astellas Pharma, Tokyo, Japan), and rapamycin (Rapamune, Pfizer Pharmaceutical Korea, Seoul, Korea) were administered daily.

### Flow Cytometric Analysis

The antibodies used in flow cytometric analysis are described in Table A.1. 7-Aminoactinomycin D (7-AAD; BD Biosciences, San Diego, CA, USA) was added to stain the dead cells. Flow cytometry was performed using an Attune NxT Flow Cytometer (Thermo Fisher Scientific). Data were analyzed using the FlowJo software (Tree Star, Ashland, OR, USA).

### Real-time Polymerase Chain Reaction

Real time polymerase chain reaction for the heart allograft tissue was performed; detailed information, including the corresponding primers used, is described in Table A.2 and A.1. Supplemental Methods. The mRNA levels of IL-1β, IL-6, and IFN-γ genes were normalized to that of glyceraldehyde 3-phosphate dehydrogenase (Gapdh) and expressed as the relative expression compared to that in the phosphate-buffered saline (PBS) group.

### Histologic Analysis

Hematoxylin and eosin staining was performed for heart allograft tissues. Immunofluorescence staining was also performed for C4d, Myc, and CD45.1 with 4,6-diamidino-2-phenylindole (DAPI, Sigma-Aldrich). Detailed information, including the antibodies used in this analysis, is described in A.1. Supplemental Methods.

### Statistical Analysis

Data are shown as the mean±standard error of the mean and were analyzed by two-tailed Student's t test. Graft survival were analyzed by the log-rank test. P<0.05 was considered as statistically significant. All analyses were performed using GraphPad Prism (v. 7.0; GraphPad Software, La Jolla, CA, USA).

### Results & Discussion

Selection of anti-C4d antibodies from combinatorial scFv-displayed phage libraries after chicken immunization

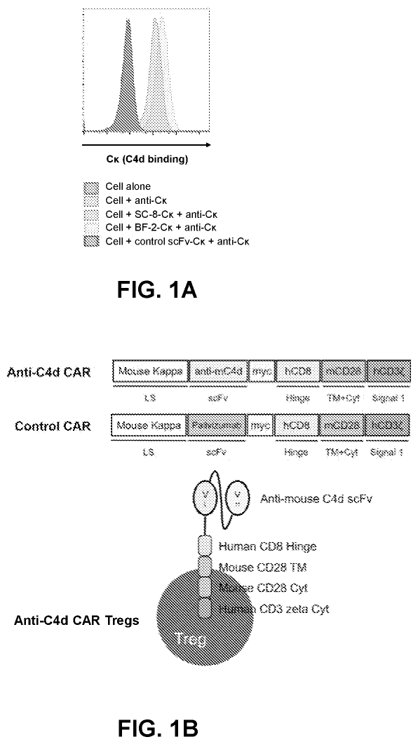

Through the phage enzyme immunoassay with bio-panned scFv-displayed phage libraries, several reactive clones were identified as candidate clones. Two clones (SC-8, BF-2) which showed good binding affinity for C4d and the BF-2 clone were selected for further study based on its binding activity and expression level (FIG. 1A).

### Construction of anti-C4d CAR Retroviral Vector

Retroviral vectors containing anti-C4d CAR were generated by cloning anti-C4d scFv into different regions of CD8, CD28, and CD3 in a second-generation CAR structure (FIG. 1B). A control CAR vector containing palivizumab scFv was also constructed (FIG. 1B).

### Generation and Phenotypes of anti-C4d CAR Tregs

Anti-C4d and control CAR Tregs, were generated according to the protocol in FIG. 2A. Both CAR Tregs expressed CAR expression (Myc+) and showed good viability (7-AAD-) and preserved forkhead box P3 (Foxp3) expression, whereas NT Tregs did not express Myc (FIG. 2B). Both CAR Tregs expressed Foxp3, CD25, cytotoxic T-lymphocyte-associated protein 4 (CTLA-4), latency-associated peptide (LAP), and glucocorticoid-induced tumor necrosis factor receptor-related protein (GITR) to a similar extent as NT Tregs (FIG. 2C), suggesting that the immunosuppressive function-associated molecules in Tregs were well-preserved in the anti-C4d CAR Tregs.

### Activation of Anti-C4d CAR Tregs by Specific Binding to C4d

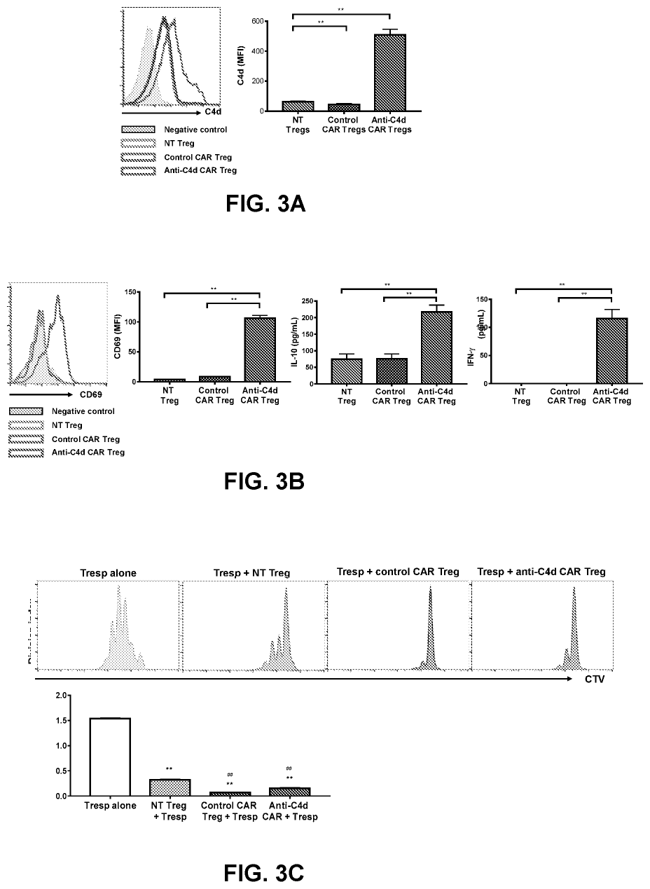

Soluble C4d-human Fc successfully bound to anti-C4d CAR Tregs, whereas it did not bind to control CAR Tregs or NT Tregs (FIG. 3A). Moreover, anti-C4d CAR Tregs upregulated CD69 expression and secreted much more IL-10 and IFN-γ in response to C4d binding, than both control CAR Tregs and NT Tregs (P<0.01, FIG. 3B). These data show that anti-C4d CAR Tregs can specifically bind to C4d and are activated by their binding to C4d.

### In Vitro Immunosuppressive Activity of Anti-C4d CAR Tregs

All three groups of Tregs suppressed T cell proliferation, although both CAR Tregs had slightly stronger suppressive effects than NT Tregs (P<0.05, FIG. 3C). These results indicate that anti-C4d CAR Tregs are functionally active Tregs and exhibit the immunosuppressive activity of Tregs.

### Suppressive Activity of anti-C4d CAR Tregs Against Heart Allograft Rejection in ABOi Heart Transplantation

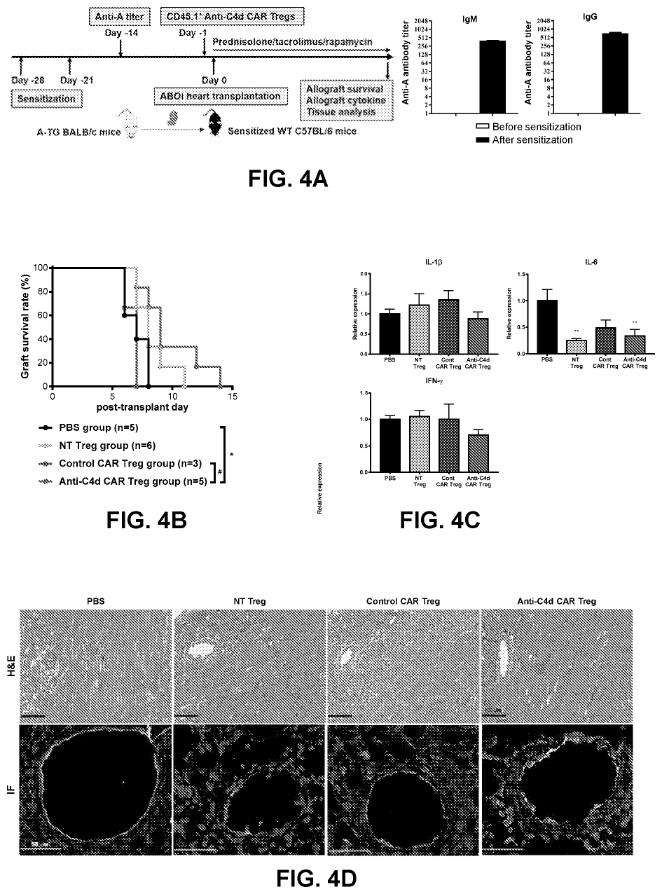

FIG. 4A shows the in vivo immunosuppressive activity of anti-C4d CAR Tregs against ABMR in ABOi heart transplantation. Sensitized recipients developed high titers of anti-A IgM and IgG before transplantation (FIG. 4A). Anti-C4d CAR Tregs significantly prolonged the ABOi heart allograft survival rate compared to the PBS control and control CAR Tregs (P<0.05, FIG. 4B). When the expression of proinflammatory cytokines in heart allografts was compared, the anti-C4d CAR Treg group showed lower IL-6 expression than the PBS control group (P<0.01, FIG. 4C); however, there was no difference between the anti-C4d CAR Treg group and NT Treg group.

Histologic examination showed perivascular inflammation and C4d deposition, indicating that ABMR indeed occurred in ABOi heart transplantation (FIG. 4D). Infiltration of CD45.1+Myc+ anti-C4d CAR Tregs around C4d+ endothelial cells was markedly observed in immunofluorescence images (FIG. 4D). However, the protective effects of CAR Tregs against tissue injury did not appear to be remarkable, possibly because all tissue studies were performed using procured terminal samples obtained after graft failure.

### Discussion

The data presented above demonstrates that anti-C4d CAR Tregs were activated by specific binding to C4d and effectively suppressed in vitro proliferation of T cells. Furthermore, anti-C4d CAR Tregs suppressed ABMR after ABOi heart transplantation and significantly prolonged ABOi heart allograft survival.

To date, anti-HLA-A2 CAR Tregs are the only CAR Tregs applied in the transplantation field and have shown good immunosuppressive effects on allograft rejection.⁶⁻⁸ However, anti-HLA-A2 CAR Tregs targeting donor-specific HLA, cannot cover all donor-recipient pairs. In contrast, the anti-C4d CAR Tregs target C4d, a well-known ABMR-associated molecule and can be used to treat most ABMR regardless of the HLA combinations of the donors and recipients. One potential limitation of anti-C4d CAR Tregs is their low ability to suppress C4d-negative ABMR.²²

On the other hand, anti-C4d CAR Tregs may prevent ABMR in ABOi transplantation by infiltrating C4d+ ABOi allografts, as C4d deposition occurs in most ABOi allografts with or without ABMR via the mechanisms of accommodation unique to ABOi transplantation.^{10,11,21} Consistent with this hypothesis, that data presented herein demonstrates that anti-C4d CAR Tregs significantly prolonged ABOi allograft survival.

The data described herein contribute to CAR Treg therapy and controlling allograft rejection in the transplantation field by providing a new type of regulatory T cell that targets C4d for both ABMR and ABOi transplantation. The results described above demonstrate for the first time the phenotypes and immunosuppressive effects of anti-C4d CAR Tregs against allograft rejection using a murine ABOi heart transplantation model.

In summary, this example describes anti-C4d CAR Tregs that increase ABOi heart allograft survival by suppressing ABMR and are therefore promising for application in human transplantation.

### Retroviral Vector Transfection and Retrovirus Packaging

The retroviral construct was transfected into the Phoenix GP (ATCC, Manassas, VA, USA) cell line, along with the pMD2.G plasmid (ATCC) containing DNA encoding the vesicular stomatitis Indiana virus G protein (VSV-G) as a virus envelope protein. At 48 h after transfection using Lipofectamine 3000 (Invitrogen, Carlsbad, CA, USA), the supernatant containing the VSV-G pseudo typed retrovirus was harvested and directly incubated with a Phoenix Eco (ATCC) cell line for infection with the retrovirus.

### Activation Assay for CAR Tregs

For the activation assay, CD20⁺ Raji cells were incubated with rituximab-mIgG2a (3 μg/mL), as previously described.¹⁹ After anti-mouse C5 antibody (200 nM, ImmunAbs, Seoul, Korea) was mixed with 5% NSG mouse serum (Chemon, Seoul, Korea), the mixture was added to rituximab-pretreated Raji cells to deposit C4d on Raji cells without cellular injury. Next, C4d⁺ Raji cells were co-cultured for 48 h with NT Tregs, control CAR Tregs, or anti-C4d CAR Tregs. Expression of CD69 in Tregs and secretion of interleukin (IL)-10 and interferon-γ (IFN-γ) were measured by flow cytometry and enzyme-linked immunosorbent assay, respectively.

### Heart Transplantation and Immunosuppressive Regimens

Wild type C57BL/6J mice were sensitized on day -21 and on day -14 by human blood group A antigen, and the serum titers of anti-A IgM and IgG were measured on day -7 by flow cytometry, as previously described.²¹ Prednisolone (1 mg/kg/day, Yuhan, Seoul, Korea), tacrolimus (Advagraf, 1 mg/kg/day, Astellas Pharma, Tokyo, Japan), and rapamycin (Rapamune, 1 mg/kg/day, Pfizer Pharmaceutical Korea, Seoul, Korea) were daily administered. The occurrence of heart allograft rejection was considered as a palpation score of 0.

### Real-time Polymerase Chain Reaction

Heart allograft tissue was homogenized with Trizol reagent (Thermo Fisher Scientific, Waltham, MA, USA), and RNA was reverse-transcribed into cDNA using Superscript II reverse transcriptase. Each reaction mixture was comprised of 2×SYBR Green PCR master mix (Applied Biosystems, Foster City, CA, USA) and 10 pmol/μL of corresponding primers (Table A.2). Analysis of real-time PCR was performed using a QuantStudio (v.3.o; Thermo Fisher Scientific).

### Histologic Analysis

The heart allografts were fixed in 4% paraformaldehyde for 24 h and paraffin-embedded sections (4 μm) were stained with hematoxylin and eosin. For immunofluorescence staining, cyostat sections (4 μm thickness) were stained with rabbit anti-mouse C4d (1:100, polyclonal, Hycult Biotech, Plymouth Meeting, PA, USA) and rat anti-mouse myc (1:200, clone 9E10, Abcam, Cambridge, UK) overnight 4° C. Next, the sections were incubated with donkey anti-rabbit IgG-Alexa Fluor 488 and goat anti-rat IgG-Alexa Fluor 647 (Thermo Fisher Scientific) for 2 hat 37° C. The anti-mouse CD45.1—Alexa Fluor 594 (clone ly-5.1, Biolegend, San Diego, CA, USA) were incubated for 2 h at 37° C., and nuclear DNA was visualized with 4,6-diamidino-2-phenylindole (DAPI, Sigma-Aldrich, St. Louis, MO, USA). The images were acquired on a Leica TCS Sp8 confocal laser scanning microscope (Wetzlar, Germany) and exported using LAS AF lite (Leica).

TABLE A
1. Antibody information used for flow cytometric analysis

| Antibody | clone | Fluorescence | Vendor |
|---|---|---|---|
| Human C-Kappa | TB28-2 | APC | BD Biosciences |
| Human IgG Fc | | FITC | Thermo Fisher |
| | | | Scientific Inc. |
| CD25 | PC61 | PE | BD Biosciences |
| CD4 | GK1.5 | PE-cy7 | eBioscience |
| CD62L | MEL-14 | APC-cy7 | BD Biosciences |
| Foxp3 | FJK-16S | FITC | eBioscience |
| CTLA-4 | UC10-4B9 | APC | Biolegend |
| LAP (TGF-β1) | TW7-16B4 | Brilliant | Biolegend |
| | | Violet 421 | |
| GITR | DTA-1 | APC | Biolegend |
| CD45.1 | A20 | APC-cy7 | Biolegend |
| CD45.2 | 104 | APC | Biolegend |

APC, Allophycocyanin; Foxp3, forkhead box P3; CTLA-4, cytotoxic T-lymphocyte-associated protein 4; Cy7, Cyanine7; FITC, Fluorescein Isothiocyanate; GITR, glucocorticoid-induced tumor necrosis factor receptor-related protein; LAP, latency-associated peptide; PE, phycoerythrin.

TABLE A.2
Primer sets used for real-time reverse transcription-
polymerase chain reaction

| | | Annealing |
|---|---|---|
| Gene | Primer sequence (5′-3′) | temperature (° C.) |
| IL-1β | F: ACTCATTGTGGCTGTGGAGA (SEQ | 60 |
| | ID NO: 27) | |
| | R: TTGTTCATCTCGGAGCCTGT (SEQ | |
| | ID NO: 28) | |
| IL-6 | F: CTGGGGATGTCTGTAGCTCA (SEQ | 60 |
| | ID NO: 29) | |
| | R: CTGTGAAGTCTCCTCTCCGG (SEQ | |
| | ID NO: 30) | |
| IFN-γ | F: GATTGCGGGGTTGTATCTGG (SEQ | 60 |
| | ID NO: 31) | |
| | R: GCTTTCTTTCAGGGACAGCC (SEQ | |
| | ID NO: 32) | |
| GAPDH | F: CAACTCCCACTCTTCCACCT (SEQ | 60 |
| | ID NO: 33) | |
| | R: GAGTTGGGATAGGGCCTCTC (SEQ | |
| | ID NO: 34) | |

F, forward; GAPDH, glyceraldehyde 3-phosphate dehydrogenase; IFN-γ, interferon-y; IL-1β, interleukin-1β; IL-6, interleukin-6; R, reverse

### Example 2

This example describes representative methods for generating regulatory T cells that express anti-C4d CAR.

Generation of mouse anti-C4d CAR Treg

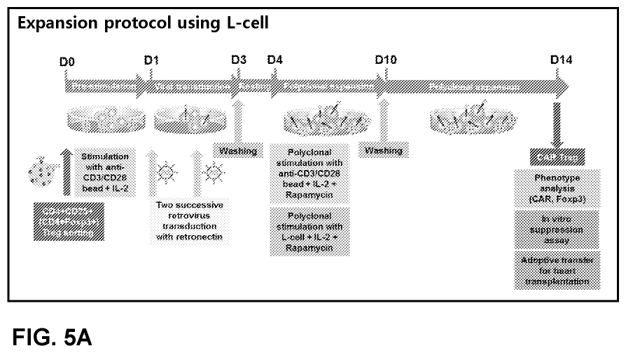

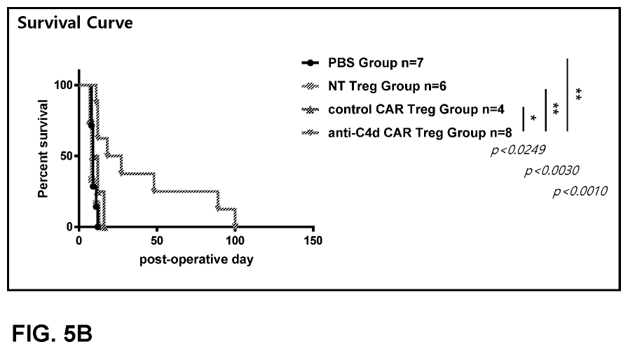

CD62L+CD4+CD25+ T cells were isolated from spleens and lymph nodes of C57BL/6J mice by sorting using FACS Aria II (BD Biosciences, San Diego, CA). Sorted Tregs were stimulated by DYNABEADS™ Mouse T-Activator CD3/CD28 (bead to cell, 1:1) and IL-2 (4,000 IU) for one day. Then, these cells were transduced with retrovirus using retronection reagent inoculated 3000 rpm, 32° C. for 90 min on two consecutive days. After washing the retrovirus using centrifuge 1500 rpm, 3 min on day 3, transduced Tregs were stimulated by L cells expressing CD86/CD64 with anti-CD3 mAbs in the presence of IL-2 and Rapamycin (100 nM) by day 7, when second round of stimulation is applied to CAR Tregs by day 13 (see FIG. 5A). As a control, nontransduced Tregs (NT Tregs) were stimulated in the same way except viral transduction. CAR's transduction efficiency was confirmed as FACS by staining Myc tag.

Generation of human anti-C4d CAR Treg

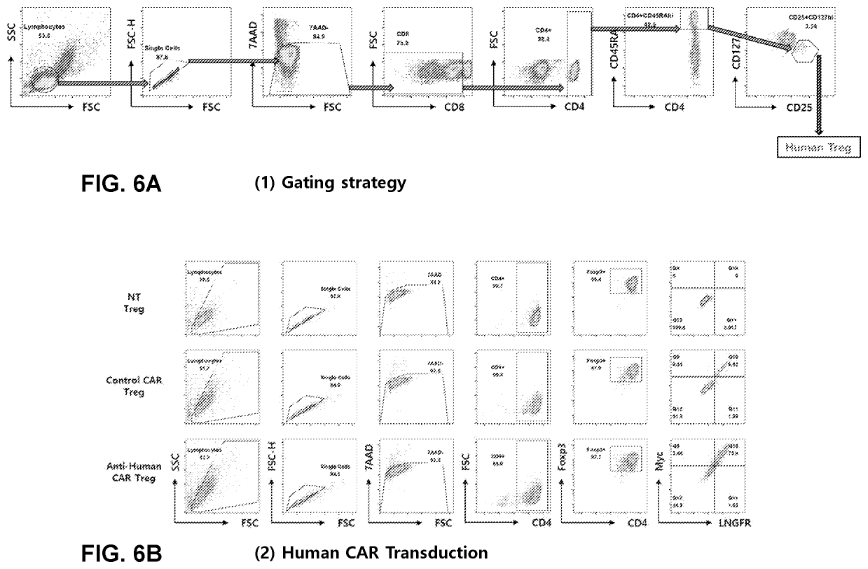

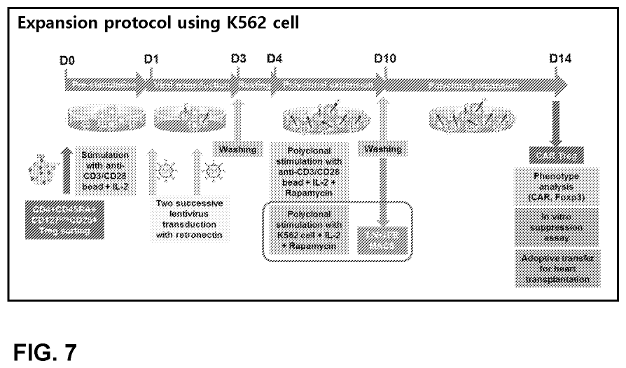

Human CD4+ T cells were isolated from human peripheral blood mononuclear cells (PBMCs) via Mojosort human CD4 T cell isolation kit. CD8-CD4+CD45RA+CD127lowCD25+ T cells were purified by fluorescence-assisted cell sorting using FACS Aria II (BD Biosciences, San Diego, CA). See FIG. 6A. Sorted Tregs were stimulated by DYNABEADS™ Mouse T-Activator CD3/CD28 (bead to cell, 1:1) and IL-2 (4,000 IU) for one day. Then, these cells were transduced with letrovirus using polybrene (6 ug/ml) reagent inoculated 25000 rpm, 25° C. for 40 min on two consecutive days. After washing the retrovirus using centrifuge 1500 rpm, 3 min on day 3, transduced Tregs were stimulated by K562 cells expressed CD86/CD64 with anti-CD3 mAbs in the presence of IL-2 and Rapamycin (100 nM) by day 7, when second round of stimulation is applied to CAR Tregs by day 13 (see FIG. 7). As a control, nontransduced Tregs (NT Tregs) were stimulated in the same way except viral transduction.

### Example 3

This example demonstrates that anti-mouse C4d CAR Tregs increase heart allograft survival rates in ABOi heart transplantation, as described in Example 1 and FIG. 4 above. As shown in FIG. 5B, anti-mouse C4d CAR Tregs significantly prolonged the ABOi heart allograft survival rate compared to the PBS control (P<0.001), NT regs (P<0.003), and control CAR Tregs (P<0.025).

### References

1. Koo T Y, Yang J. Current progress in ABO-incompatible kidney transplantation. Kidney Res Clin Pract. 2015;34(3):170-179.

2. Okumi M, Toki D, Nozaki T, et al. ABO-Incompatible Living Kidney Transplants: Evolution of Outcomes and Immunosuppressive Management. Am J Transplant.

2016;16(3):886-896.

3. de Weerd A E, Betjes MGH. ABO-Incompatible Kidney Transplant Outcomes: A Meta-Analysis. Clin J Am Soc Nephrol. 2018;13(8):1234-1243.

4. Tang Q, Bluestone J A. Regulatory T-cell therapy in transplantation: moving to the clinic. Cold Spring Harb Perspect Med. 2013;3(11).

5. Singh A K, McGuirk J P. CAR T cells: continuation in a revolution of immunotherapy. Lancet Oncol. 2020;21(3):e168-e178.

6. MacDonald K G, Hoeppli R E, Huang Q, et al. Alloantigen-specific regulatory T cells generated with a chimeric antigen receptor. J Clin Invest. 2016;126(4):1413-1424.

7. Noyan F, Zimmermann K, Hardtke-Wolenski M, et al. Prevention of Allograft Rejection by Use of Regulatory T Cells With an MHC-Specific Chimeric Antigen Receptor. Am J Transplant. 2017;17(4):917-930.

8. Boardman D A, Philippeos C, Fruhwirth G O, et al. Expression of a Chimeric Antigen Receptor Specific for Donor HLA Class I Enhances the Potency of Human Regulatory T Cells in Preventing Human Skin Transplant Rejection. Am J Transplant. 2017;17(4):931-943.

9. Haas M, Sis B, Racusen L C, et al. Banff 2013 meeting report: inclusion of c4d-negative antibody-mediated rejection and antibody-associated arterial lesions. Am J Transplant. 2014; 14(2):272-283.

10. Garcia de Mattos Barbosa M, Cascalho M, Platt J L. Accommodation in ABO-incompatible organ transplants. Xenotransplantation. 2018;25(3):e12418.

11. Setoguchi K, Ishida H, Shimmura H, et al. Analysis of renal transplant protocol biopsies in ABO-incompatible kidney transplantation. Am J Transplant. 2008;8(1):86-94.

12. Haas M, Rahman M R, Racusen L C, et al. C4d and C3d staining in biopsies of ABO- and HLA-incompatible renal allografts: correlation with histologic findings. Am J Transplant. 2006;6(8):1829-1840.

13. Motyka B, Fisicaro N, Wang S I, et al. Antibody-Mediated Rejection in a Blood Group A-Transgenic Mouse Model of ABO-Incompatible Heart Transplantation. Transplantation. 2016;100(6): 1228-1237.

14. Lee M S, Lee J C, Choi C Y, Chung J. Production and characterization of monoclonal antibody to botulinum neurotoxin type B light chain by phage display. Hybridoma (Larchmt). 2008;27(1): 18-24.

15. Lee Y, Kim H, Chung J. An antibody reactive to the Gly63-Lys68 epitope of NT-proBNP exhibits 0-glycosylation-independent binding. Exp Mol Med. 2014;46:e114.

16. Barbas III CF BD, Scott J K, Silverman G J. Phage display- a laboratory manual. New York: Cold Spring Harbor Laboratory Press; 2001.

17. Yang W, Yoon A, Lee S, Kim S, Han J, Chung J. Next-generation sequencing enables the discovery of more diverse positive clones from a phage-displayed antibody library. Exp Mol Med. 2017;49(3):e308.

18. Yoon S, Kim Y H, Kang S H, et al. Bispecific Her2 x cotinine antibody in combination with cotinine-(histidine)2-iodine for the pre-targeting of Her2-positive breast cancer xenografts. J Cancer Res Clin Oncol. 2014;140(2):227-233.

19. Dechant M, Weisner W, Berger S, et al. Complement-dependent tumor cell lysis triggered by combinations of epidermal growth factor receptor antibodies. Cancer Res. 2008;68(13):4998-5003.

20. Brender C, Tannahill G M, Jenkins B J, et al. Suppressor of cytokine signaling 3 regulates CD8 T-cell proliferation by inhibition of interleukins 6 and 27. Blood. 2007;110(7):2528-2536.

21. Park S, Lee J-G, Jang J Y, et al. Induction of Accommodation by Anti—complement Component 5 Antibody-based Immunosuppression in ABO-incompatible Heart Transplantation. Transplantation. 2019;103(9):e248-e255.

22. Orandi B J, Alachkar N, Kraus E S, et al. Presentation and Outcomes of C4d-Negative Antibody-Mediated Rejection After Kidney Transplantation. Am J Transplant. 2016;16(1):213-220.

23. Wu G D, He Y, Chai N N, et al. Anti-CD20 antibody suppresses anti-HLA antibody formation in a HLA-A2 transgenic mouse model of sensitization. Transpl Immunol. 2008;19(3-4):178-186.

It is understood that the examples and embodiments described herein are for illustrative purposes only and that various modifications or changes in light thereof will be suggested to persons skilled in the art and are to be included within the spirit and purview of this application and scope of the appended claims. All publications, Genbank accession numbers, patents, and patent applications cited herein are hereby incorporated by reference in their entirety for all purposes.

1. A genetically modified regulatory T cell (Treg) comprising an antigen binding protein (ABP) that specifically binds complement component 4d (C4d).

2. The regulatory T cell of claim 1, wherein the antigen binding protein comprises a chimeric antigen receptor (CAR).

3. The regulatory T cell of claim 2, wherein the CAR comprises a scFv that specifically binds C4d.

4. The regulatory T cell of claim 1, wherein the ABP, CAR or scFv comprises:
a light chain variable region (VL) comprising:
(i) a light chain complementary determining region (LCDR) 1 comprising the amino acid sequence SGSSGSYG (SEQ ID NO: 68), SGGGRWYG (SEQ ID NO: 84), or SGGGSYYG (SEQ ID NO: 43); or a variant LCDR1 in which 1, 2, 3, 4, or 5 amino acids are substituted relative to the sequence of SEQ ID NOs: 43, 68, or 84;
(ii) an LCDR2 comprising the amino acid sequence YNDKRPS (SEQ ID NO: 69), HANTKRPS (SEQ ID NO: 85), or SNNKRPS (SEQ ID NO: 44); or a variant LCDR2 in which 1, 2, 3, 4, or 5 amino acids are substituted relative to the sequence of SEQ ID NOs: 44, 69, or 85; and
(iii) an LCDR3 comprising the amino acid sequence GSEDSSYVGV (SEQ ID NO: 70), GSGDSSTDSGI (SEQ ID NO: 86), or GSYDSNAGI (SEQ ID NO: 45); or a variant LCDR3 in which 1, 2, 3, 4, or 5 amino acids are substituted relative to the sequence of SEQ ID NOs: 45, 70, or 86; and
a heavy chain variable region (VH) comprising:
(i) heavy chain complementary determining region 1 (HCDR1) comprising the amino acid sequence SYALE (SEQ ID NO: 71), DRAMH (SEQ ID NO: 87), or SYAMG (SEQ ID NO: 48); or a variant HCDR1 in which 1, 2, 3, 4, or 5 amino acids are substituted relative to the sequence of SEQ ID NOs: 48, 71, or 87;
(ii) an HCDR2 comprising the amino acid sequence GISSSGSGTNYGSAVKG (SEQ ID NO: 72), GIYSSGRYTGYGSAVKG (SEQ ID NO: 88), or EISGSGTSTYYGPAVKG (SEQ ID NO: 49); or a variant HCDR2 in which 1, 2, 3, 4, or 5 amino acids are substituted relative to the sequence of SEQ ID NOs: 49, 72, or 88; and
(iii) an HCDR3 comprising the amino acid sequence AYGYVDAYGIDA (SEQ ID NO: 73), AGSIYCGYADVACIDA (SEQ ID NO: 89), or CTRGGGAGSYIDA (SEQ ID NO: 50); or a variant HCDR3 in which 1, 2, 3, 4, or 5 amino acids are substituted relative to the sequence of SEQ ID NOs: 50, 73, or 89.

5. The regulatory T cell of claim 4, wherein the VL comprises an amino acid sequence having at least 95% identity to
LTQPSSVSANPGGTVEITCSGSSGSYGWYQQKSPGSAPVTVIYYNDKRPSDIPSRFSGSKS GSTATLTITGVQAEDEAVYFCGSEDSSYVGVFGAGTTLTVL (SEQ ID NO: 2)
LTQPSSVSANPGETVKITCSGGGRWYGWYQQKSPGSAPVTLIHANTKRPSNIPSRFSGSL SGSTSTLTISGVQAEDEAVYFCGSGDSSTDSGIFGAGTTLTVL (SEQ ID NO: 6), or
LTQPSSVSANPGETVEITCSGGGSYYGWYQQKSPGSAPVTVIYSNNKRPSDIPSRFSGSKS GSTSTLTITGVQADDEAVYYCGSYDSNAGIFGAGTTLTVL (SEQ ID NO: 42); and the VH comprises an amino acid sequence having at least 95% identity to
AVTLDESGGGLQTPGGTLSLVCKGSGFTFRSYALEWVRQAPGKGLEYVAGISSSGSGTN YGSAVKGRATISRDNGQSTVRLQLNNLRAEDTGTYYCAKSAYGYVDAYGIDAWGHGT EVIVSSTS (SEQ ID NO: 4)
AVTLDESGGGLQTPGGALSLVCKASGFSFSDRAMHWVRQAPGKGLEWVAGIYSSGRYT GYGSAVKGRATISRDNGQSTVRLQLNNLRAEDTGTYYCAKAGSIYCGYADVACIDAWG HGTEVIVSSTS (SEQ ID NO: 8), or
AVTLDESGGGLQTPGGALSLVCKASGFTFSSYAMGWMRQAPGKGLDFVAEISGSGTST YYGPAVKGRATISRDNGRSTVRLQLNNLRAEDTGTYFCTRGGGAGSYIDAWGHGTEVI VSSTS (SEQ ID NO: 47).

6-11. (canceled)

12. The regulatory T cell of claim 3, wherein the scFv comprises an amino acid sequence having at least 80% sequence identity to:
i)
(SEQ ID NO: 67)
LTQPSSVSANPGGTVEITCSGSSGSYGWYQQKSPGSAPVTVIYYNDKRPSDIPSR
FSGSKSGSTATLTITGVQAEDEAVYFCGSEDSSYVGVFGAGTTLTVLGQSSRSSGGGGSS
GGGGSAVTLDESGGGLQTPGGTLSLVCKGSGFTFRSYALEWVRQAPGKGLEYVAGISSS
GSGTNYGSAVKGRATISRDNGQSTVRLQLNNLRAEDTGTYYCAKSAYGYVDAYGIDA
WGHGTEVIVSSTS;
ii)
(SEQ ID NO: 83)
LTQPSSVSANPGETVKITCSGGGRWYGWYQQKSPGSAPVTLIHANTKRPSNIP
SRFSGSLSGSTSTLTISGVQAEDEAVYFCGSGDSSTDSGIFGAGTTLTVLGQSSRSSGGGG
SSGGGGSAVTLDESGGGLQTPGGALSLVCKASGFSFSDRAMHWVRQAPGKGLEWVAGI
YSSGRYTGYGSAVKGRATISRDNGQSTVRLQLNNLRAEDTGTYYCAKAGSIYCGYADV
ACIDAWGHGTEVIVSST;
or
iii)
(SEQ ID NO: 36)
LTQPSSVSANPGETVEITCSGGGSYYGWYQQKSPGSAPVTVIYSNNKRPSDIPS
RFSGSKSGSTSTLTITGVQADDEAVYYCGSYDSNAGIFGAGTTLTVLGQSSRSSGGGGSS
GGGGSAVTLDESGGGLQTPGGALSLVCKASGFTFSSYAMGWMRQAPGKGLDFVAEISG
SGTSTYYGPAVKGRATISRDNGRSTVRLQLNNLRAEDTGTYFCTRGGGAGSYIDAWGH
GTEVIVSSTS.

13. The regulatory T cell of claim 2, wherein the CAR comprises a leader sequence.

14. (canceled)

15. The regulatory T cell of claim 13, wherein the CAR comprises a human CD8 hinge region.

16. (canceled)

17. The regulatory T cell of claim 2, wherein the CAR comprises a CD28 transmembrane domain.

18. The regulatory T cell of claim 2, wherein the CAR comprises a CD28 cytoplasmic domain or a CD3 zeta cytoplasmic domain.

19-21. (canceled)

22. The regulatory T cell of claim 2, wherein the CAR comprises a c-myc tag.

23. A regulatory T cell comprising a nucleic acid encoding the antigen binding protein of claim 1.

24. A vector comprising a nucleic acid encoding the antigen binding protein of claim 1.

25. (canceled)

26. A cell comprising the vector of claim 24.

27. (canceled)

28. A method for producing the regulatory T cell of claim 1, comprising transfecting or transducing the regulatory T cell with a vector comprising a nucleic acid encoding the antigen binding protein of claim 1, and selecting a Treg that expresses the antigen binding protein.

29. An in vitro method for inducing an immune response, the method comprising contacting a regulatory T cell of claim 1 with C4d antigen.

30. (canceled)

31. An in vitro method for suppressing T cell proliferation, the method comprising culturing a regulatory T cell of claim 1 with an activated effector T cell and determining a decrease in proliferation of the effector T cell.

32. A method for suppressing antibody-mediated rejection (ABMR) in a subject receiving a transplant, comprising administering a therapeutically effective amount of the regulatory T cell of claim 1 to a subject.

33. The method of claim 32, wherein the transplant is an allograft.

34. The method of claim 33, wherein the allograft is an ABO blood group-incompatible (ABOi) allograft.

35. (canceled)
